SEQUENCE LISTING (7 sequences)

SEQ ID NO: 1 - DNA, 15 bp, ARTIFICIAL
  APTAMER
ggttggtgtggttgg

SEQ ID NO: 2 - DNA, 27 bp, ARTIFICIAL
  APTAMER
acccgtggtagggtaggatggggtggt

SEQ ID NO: 3 - DNA, 15 bp, ARTIFICIAL
  APTAMER
ggttggtgtggttgg

SEQ ID NO: 4 - DNA, 15 bp, ARTIFICIAL
  APTAMER
ggttggtgtggttgg

SEQ ID NO: 5 - DNA, 27 bp, ARTIFICIAL
  APTAMER
acccgtggtagggtaggatggggtggt

SEQ ID NO: 6 - DNA, 27 bp, ARTIFICIAL
  APTAMER
acccgtggtagggtaggatggggtggt

SEQ ID NO: 7 - protein, 12 aa, ARTIFICIAL
  SULFATED HIRUDIN FRAGMENT
GDFEEIPEEYLQ

(19) United States
(12) Patent Application Publication
(10) Pub. No.: US 2009/0208936 A1
(43) Pub. Date: Aug. 20, 2009

(54) IDENTIFICATION OF MOLECULAR INTERACTIONS AND THERAPEUTIC USES THEREOF

(75) Inventors: Weihong Tan, Gainesville, FL (US); Zehui Cao, Urbana, IL (US)

Correspondence Address: AKERMAN SENTERFITT, P.O. BOX 3188, WEST PALM BEACH, FL, 33402-3188, US

(73) Assignee: Univeristy of Florida Research Foundations Inc., Gainesville, FL (US)

(21) Appl. No.: 11/813,337

(22) PCT Filed: Jan. 4, 2006
(86) PCT No.: PCT/US06/00167

§ 371 Date: May 15, 2008

Related U.S. Application Data
(60) Provisional application No. 60/641,296, filed on Jan. 4, 2005.

(51) Int. Cl. C12Q 1/68; C07H 21/04

(52) U.S. Cl. 435/6; 536/23.1

(57) ABSTRACT

We report the real-time monitoring of protein-protein interactions without labeling either of the two interacting proteins, posing minimum effects on the binding properties of the proteins. In particular, the methods provide protein/aptamer complexes to probe the interactions in a competitive assay where the binding of an aptamer to its target protein is altered by a second protein that interacts with the target protein. Two signal transduction strategies, fluorescence resonance energy transfer (FRET) and fluorescence anisotropy, are described.

### FIELD OF THE INVENTION

The invention relates to the field of protein-protein interactions for diagnosis of disease and identification of therapeutic drugs. In particular, the invention describes the real-time monitoring of protein-protein interactions without labeling either of the two interacting proteins, posing minimum effects on the binding properties of the proteins.

### BACKGROUND

The functions of living cells are mostly executed and regulated by proteins. The important roles of proteins are often realized through interactions between two or more proteins. As an example, growth factor proteins interact with their receptors on the cell membrane to regulate the proliferation of the cells. In order to understand how cells fulfill their functions and how they react to changes in the environments, it is necessary to gain insight into how proteins interact with each other under different conditions. However, many commonly used techniques based on molecular separation such as gel electrophoresis and capillary electrophoresis (CE) lack the ability of real-time analysis in homogeneous solutions. A more recent development in protein-protein interactions is the yeast two-hybrid system that was first reported in 1989. Based on transcription activated in yeast nuclei by protein-protein interactions, it has been widely used to study protein functions, and recently adapted to map protein interactions on a proteome-wide scale. However, it is usually not done in real time and involves labor-intensive procedures to fuse the two proteins into a DNA-binding domain and an activation domain.

Another technique capable of protein-protein interaction monitoring is based on fluorescence resonance energy transfer (FRET), where two interacting proteins have to be dye-labeled for the energy transfer to take place. It is well known that the functions of proteins in biological systems are highly dependent on their tertiary structures. As a result, chemical modifications to proteins such as dye labeling may cause a reduction or even a loss of protein activities by either directly blocking the active binding sites or affecting the three-dimensional folding of the proteins.

Therefore, it is highly desirable to avoid any modifications of proteins when monitoring protein-protein interactions in order to obtain the most “true-to-life” information.

### SUMMARY

A label-free target molecule detection system and methods provide a versatile composition for real-time molecular interaction study based on aptamers. In particular, molecular beacon aptamers are described comprising the superior specificity of aptamers for proteins and the excellent signal transduction mechanism of molecular beacons. Furthermore, the invention provides methods for detection of molecular interactions, wherein the target protein lacks a label.

In a preferred embodiment, new compositions, systems, and methods for simultaneously detecting the presence and quantity of one or more different compounds in a sample use novel nucleic acid molecules. Nucleic acids have been shown to be capable of specifically binding with high affinity to non-nucleotide target molecules, such as proteins, small organic molecules, or inorganic molecules. These nucleic acids are commonly referred to as aptamers. An aptamer can be either an RNA or a DNA composed of naturally occurring or modified nucleotides.

In a preferred embodiment, the invention provides compositions comprising aptamers for detection of protein-protein interaction and detection; for determination of protein functions; for protein and drug molecule interaction.

In a preferred embodiment, aptamer compositions are identified by any one of SEQ ID NO's: 1-4 variants or fragments thereof.

In another preferred embodiment, the invention provides for aptamer compositions which are about 45% homologous to any one of SEQ ID NO's 1-4; preferably, the aptamer compositions are about 55% homologous to any one of SEQ ID NO's 1-4; preferably, the aptamer compositions are about 65% homologous to any one of SEQ ID NO's 1-4; preferably, the aptamer compositions are about 75% homologous to any one of SEQ ID NO's 1-4; preferably, the aptamer compositions are about 85% homologous to any one of SEQ ID NO's 1-4; preferably, the aptamer compositions are 90%, 91%, 92%, 93%, 94%, 95%, 96%, 97%, 98%; 99% and 99.9% homologous to any one of SEQ ID NO's 1-4.

In another preferred embodiment, the invention provides methods for real time protein detection. Preferably, fluorophores on the aptamers are dequenched. Preferably, the assays described herein, include, the FRET-based assay which relies on direct measurements of sample fluorescence, making it highly sensitive and selective. It can also be easily adapted for binding site-specified high throughput protein interaction screening in an array format.

In another preferred embodiment, the aptamer-based competitive assay, described herein, is used for finding protein-binding targets with comparable affinities in a large array of compounds. When detection of weaker protein-protein binding is desired, it is possible to lower the aptamer's affinity towards the target protein by adding, removing or changing bases of the aptamer that are not directly involved in the aptamer/protein binding. This flexibility or tenability makes aptamers more appealing for competitive assays than antibodies.

In accordance with the invention, the aptamer competitive assay provides a method for determining interactions between proteins and other molecules such as small organic molecules, DNAs and RNAs. With aptamers being rapidly developed for a growing number of proteins, it is possible to build a large array of aptamers in protein-drug candidate interactions for large scale drug discovery, or in whole cell protein-protein interactions for disease diagnosis and functional proteomics.

In a preferred embodiment, aptamers are bioengineered such that binding of a bioengineered aptamer to a target molecule causes a change in the conformation of the bioengineered aptamer. Furthermore, one or more reporter moieties or groups are included in the bioengineered aptamers such that the change in bioengineered aptamer conformation results in a detectable change of a physical property of the reporter group (or the engineered aptamer itself). These bioengineered aptamers are referred to herein as aptamers.

Aptamers having binding regions configured to bind to different target molecules can be used in various detection methods and systems. For example the new aptamers can be used in solution-based assays, or can be attached to a solid support, e.g., at different predetermined points in a one or two-dimensional array, for use in solid-based assays. The aptamers or aptamer arrays are then exposed to the sample, such that target molecules in the sample bind to their respective aptamers. The presence of bound target molecules can be detected by measuring a change in a physical property of the aptamer reporter group, e.g., by observing a change in fluorescence efficiency of the aptamer. To assist in analyzing the sample, the new detection systems can include pattern recognition software. The software compares the target molecule binding pattern corresponding to the unknown sample with binding patterns corresponding to known compounds. From these comparisons, the software can determine the composition of the sample, or deduce information about the source of the sample. The systems can be used to detect the existence of characteristic compounds, or “molecular fingerprints,” associated with certain chemicals or conditions. For example, the systems can be used for human drug testing by detecting the presence of metabolites of particular drugs. The systems can also be used to infer the existence of a disease (e.g., cancer) by detecting the presence of compounds associated with the disease state, or for pollution monitoring by detecting compounds characteristic of the discharge of certain pollutants. Numerous other applications are also possible.

In a preferred embodiment, aptamers are designed for protein-protein interaction and detection. Preferably, aptamers are systematically selected nucleic acids that have high affinity and selectivity for their target proteins. We have developed techniques that enable label-free analysis of proteins in real time and homogeneous solutions based on the aptamers. Fluorescence steady state and polarization measurements are used for signal transduction. In steady state, the aptamer is labeled with two fluorophores that have overlapping excitation and emission spectra. When bound to the target protein, the binding-induced conformational change of the aptamer causes two fluorophores to be in close proximity and change their fluorescence intensity because of fluorescence resonance energy transfer (FRET). This process can be used to report the presence of the target protein without labeling it. By determining the fluorescence of either one of the two fluorophores, a fluorescence quenching assay or a fluorescence generating assay can be used, depending on the specific application. hi the polarization measurements, the aptamer is labeled with only one fluorophore. Binding to a much larger protein target results in a slower diffusional rotation of the aptamer and increased fluorescence anisotropy of the fluorophore.

In another preferred embodiment, aptamers are designed for protein function studies. Preferably, the aptamers are used in the competitive assay systems of the invention. In a competitive assay, the aptamer/target protein binding complex can be disrupted by a third molecule, either a protein or a drug molecule, if the third molecule can interact with the target protein. This protein-molecule interaction can be readily reflected in real time by changes in the fluorescent signals of the aptamer. By analyzing FRET or anisotropy of the dye-labeled aptamer, we have been able to monitor protein-protein and protein-small molecule interactions in homogeneous solution as well as determine the kinetics and binding sites of the interactions. All this can be done easily and quickly without labeling the protein or the third molecule, which gives us most “true-to-life” insight into protein functions based on the unaffected protein structures.

In another preferred embodiment, assays are aptamer based assays for identifying protein and drug molecules. Fluorescence assays developed herein, for protein analysis and protein function study can be easily adapted to large-scale formats, such as a 96-well array, for high-throughput protein study. Diagnoses of diseases including cancers can be carried out by identify certain protein markers that are present in the cells. By developing aptamers for different cancer marker proteins, arrays can be constructed that have the capability of sensitive multiplex cancer marker detection. Based on our aptamer assay, the analysis of the cell content conducted highly efficiently. The fluorescence signals obtained from the array generate a pattern which shows the presence of different cancer related proteins. By comparing the patterns acquired from different cell samples, cancer diagnoses may be done with great ease and accuracy.

In another preferred embodiment, aptamer based assays include dequenching assays. These assays measure dequenching of fluorophores on aptamers for real time protein detection. Protein-binding aptamer based assays are shown to be capable of sensitive protein detection in real time. The signal transduction mechanisms used in such detection include fluorescence resonance energy transfer (FRET) and fluorescence anisotropy (FA). In FRET, a fluorophore and a quencher are labeled on the aptamer and a protein-binding induced conformational change of the aptamer is required to trigger a fluorescence signal change. In FA, two polarizers are needed which causes much lower detected fluorescence intensity compared to steady state measurements.

In another preferred embodiment, fluorophores are identified which, when attached to certain positions on the aptamer, display a significant fluorescence enhancement upon protein binding. Without wishing to be bound by theory, this result may be explained that the fluorophore is quenched by the nucleic acid bases of the aptamer. Binding to the protein can alleviate the fluorophore from the quenching environments and cause a restored fluorescence. This new finding has allowed the design and construction of assays that requires only one dye on the aptamer, as in FA, while having similar sensitivity and dynamic range as in FRET. It also reduces the concern of aptamer conformational change that is necessary with FRET. The dequenching of fluorophores on aptamers (DFA) assay should provide an economical and sensitive alternative for real-time protein detection in homogeneous solutions.

In another preferred embodiment, an aptamer binds to a non-nucleic acid target molecule and administration of a candidate prey protein results in the dissociation of aptamer-bait/target molecule resulting in a change in fluorescence, the measure of which identifies the candidate prey molecule as a molecule that binds the target/bait molecule. The aptamers comprise a first reporter moiety which can be an energy absorbing moiety and the second reporter moiety can be a fluorescence emitting moiety, such that when the first and second reporter moieties are in sufficiently close proximity, the absorbing moiety allows an energy transfer between the moieties, thereby allowing the emitting moiety to fluoresce.

In another aspect, the invention features a device for simultaneously detecting the presence of a plurality of different, non-nucleic acid target molecules in a sample. The devices include: a solid support; and a plurality of different aptamers bound to the support, each aptamer having a first end attached to the support, and a binding region that binds to a specific non-nucleic acid target molecule, wherein the binding regions of different aptamers bind to different target molecules. In these devices, the solid support can be a glass surface to which the first ends of the aptamers are covalently bound. In addition, the solid support can be a planar surface, and the aptamers can be distributed on the planar surface in a two-dimensional array. Spots of identical aptamers can be located at different points in the two-dimensional array.

The binding region of at least one of the aptamers in the device can be configured to bind to a non-nucleic acid target molecule selected from the group consisting of a protein, a small organic molecule, nucleic acid molecules, and an inorganic molecule. The aptamers can comprise RNA, DNA, modified RNA, modified RNA, or a combination thereof. In addition, each aptamer can comprise a reporter group, such as a fluorophore, for signaling binding of a target molecule to the binding region and/or a quencher.

The invention also features a method of detecting the presence or absence of one or more different target molecules in a sample, by obtaining a plurality of the new aptamers; contacting the sample to the aptamers, such that any target molecules in the sample can bind to corresponding binding regions of the aptamers; and detecting the presence of target molecules bound to the aptamers. The aptamers can be in a liquid, or can be bound to a solid support, such as a particle or a plate. In some embodiments, the aptamers emit fluorescent radiation when excited by evanescent waves.

In this method, different spots, each spot including a plurality of identical aptamers, can be distributed on the solid support in a predetermined array, and the method can further include comparing a fluorescence pattern of the sample to known fluorescence patterns, e.g., with a computer program, disposed on a computer readable medium, that includes instructions for causing a processor to compare the fluorescence pattern of the sample to a library of known fluorescence patterns; and select the combination of known fluorescence patterns that most closely matches the fluorescence pattern of the sample. The detecting step can also include detecting a change in the Raman emission frequencies of an aptamer caused when a target molecule binds to the aptamer.

In another aspect, the invention features a computer program, disposed on a computer-readable medium, for analyzing the output of an assay that determines the composition of a sample and deduces the presence or absence of known abnormal conditions, the computer program including instructions for causing a processor to: compare the assay output, e.g., an image, to a library of known outputs corresponding to subjecting samples of known composition to the assay; select a combination of known outputs that most closely matches the assay outputs; compare any deviation between the sample output and the combination of known outputs to a library of known deviations, the known deviations being caused by known abnormal conditions; and deduce the presence or absence of known abnormal conditions. For example, the known abnormal conditions can include the presence of abnormal compounds in the sample, and the presence of normal compounds in abnormal quantities.

In another preferred embodiment, the invention features a method for detecting the presence of a target molecule in a sample. Preferably, a plurality of different aptamers are bound to a support, each aptamer having a first end attached to the support, and a binding region that binds to a specific enantiomer of the target molecule, wherein the binding regions of different aptamers bind to different enantiomers of the target molecule. However, target molecules can also be determined in a solution. Aptamers can also be designed with a binding region that binds to a specific binding site of the target, wherein the binding regions of different aptamers bind to different binding sites. For example, the target can be an antigen, and the different binding sites can be different epitopes of the antigen, or the target can be a bacteria, and the different binding sites can be different surface proteins of the bacteria.

The invention further features a system for simultaneously detecting the presence of a plurality of different non-nucleic acid target molecules in a sample. The system includes a solid support (optional); a plurality of different aptamers, optionally bound to the support, each aptamer having a first end attached to the support, a binding region that binds to a specific non-nucleic acid target molecule, the binding regions of different aptamers binding to different target molecules; and a detection system that detects the presence of target molecules bound to aptamers, the detection system including a radiation source, e.g., a laser, and a detector. The system can further include an analyzer for determining the presence of target molecules in the sample based on the output of the detection system. The analyzer can also include a computer processor programmed to compare the output of the detection system to a library of known outputs corresponding to exposing samples of known composition to the aptamers on the solid support; and select a combination of known outputs that most closely matches the assay outputs. The computer processor can be further programmed to compare any deviation between the output of the detection system and the combination of known outputs to a library of known deviations, the known deviations being caused by known abnormal conditions; and deduce the presence or absence of known abnormal conditions.

In yet another aspect, the invention features a method or system for simultaneously detecting the presence or absence of one or more different target molecules in a sample using a plurality of different species of aptamers, wherein each species of aptamers has a different reporter group, a binding region that binds to a specific non-nucleic acid target molecule, and wherein the binding regions of different aptamers bind to different target molecules; and a detection system that detects the presence of target molecules bound to aptamers, the detection system being able to detect the different reporter groups. The method can also be carried out with a plurality of identical aptamers. For example, each aptamer can include a reporter such as a molecular beacon that changes fluorescence properties upon target binding. Each species of aptamer can be labeled with a different fluorescent dye to allow simultaneous detection of multiple target molecules, e.g., one species might be labeled with fluorescein and another with rhodamine. The fluorescence excitation wavelength (or spectrum) can be varied and/or the emission spectrum can be observed to simultaneously detect the presence of multiple targets.

The fluorescence measurement can be performed with a number of different instruments, including standard fluorescence spectrophotometers, or in a small volume using a high-intensity source, such a laser, high-efficiency light collection optics, such as a high-numeric aperture microscope objective, and a high-efficiency low-noise detector, such as photo-multiplier tube, a photodiode or a CCD camera.

The method can further include a computer program that includes instructions for causing the processor to compare the measured fluorescence emission or excitation spectrum with the known spectrum of each of the individual dyes to quantitatively determine the concentration of each of the target molecules in the solution.

Different aspects of the invention may include one or more of the following advantages. The aptamer-based detection systems allow the detection of a plurality of different compounds simultaneously, or high sensitivity detection of a single target in a plurality of different ways. Unlike antibodies, which are selected in an organism, the aptamers can be selected in vitro, e.g., in a test tube. This allows detection of target molecules that are toxic or immunologically inert. Furthermore, the aptamers in the detection systems have high affinities for their target molecules, allowing ultra-sensitive detection. As a result, the systems are highly specific, and can distinguish molecules that differ by as little as a single methyl or hydroxyl group. The systems also allow rapid analysis of a sample (as quickly as a few minutes), facilitating detection of unstable compounds. In addition, the reagents used in the assay are inexpensive, and the chemistry involved in performing the assay is easily automated.

The detection systems can be used in a variety of applications, including drug testing, high-sensitivity testing for the presence of bacteria or antigens, pollution monitoring, and testing for the presence or absence of a disease.

In another preferred embodiment, a method of increasing selectivity and affinity of aptamers for a target molecule comprises producing aptamers specific for a target protein; selecting aptamers that bind different epitopes on the target protein; linking the selected aptamers with a linking molecule; thereby, increasing the selectivity and affinity of the aptamers for a target molecule. Preferably, at least two aptamers specific for the target protein are linked. The aptamers can be linked by any suitable molecule, such as for example, a polyethylene glycol chain.

In a preferred embodiment, the binding association constants (on rates) of the linked aptamers is greater than the binding association constants of each individual aptamer.

In another preferred embodiment, at least one of the aptamers is labeled with a donor molecule at a 5′-end and an acceptor molecule at a 3′-end. Preferably, the donor molecule is a fluorophore molecule and the acceptor molecule is a fluorophore quenching molecule.

In a preferred embodiment, binding of the aptamers to a target/bait molecule is detected by a quenching of fluorescence as compared to a baseline fluorescence of unbound aptamer(s).

In another preferred embodiment, a method of diagnosing a disease comprises binding of an aptamer to a biomarker of disease; detecting the binding of the aptamer to the biomarker as compared to a control; and, diagnosing a disease.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and are not intended to be limiting.

Other aspects of the invention are described infra.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIGS. 1A and 1B are a schematic illustration of dye-labeled protein-binding aptamers reporting protein-protein interactions. FIG. 1A shows a dual-labeled aptamer with a fluorophore and a quencher. The folded form of the aptamer when it binds to the bait protein results in a quenched fluorescence. The bait-prey protein interaction causes release of aptamer from the bait protein, leading to a restored fluorescence; FIG. 1B shows an aptamer labeled with only one dye. When bound to the much larger bait protein, the aptamer displays slow rotational diffusion. The interaction between bait and prey proteins displaces the aptamer. The unbound aptamer has much faster rotational diffusion. The change in the rotation rate is reported by fluorescence anisotropy of the dye molecule.

FIGS. 2A and 2B are graphs showing α-thrombin binding induced relative fluorescence change of dual-labeled 15mer aptamer. FIG. 2A is a graph showing α-thrombin binding induced relative fluorescence change of dual-labeled 15mer aptamer. 6-FAM florescence intensities of 100 nM FQ-15Ap, FQ-27Ap and F-15Ap were recorded before (blue column) and after (white column) the addition of 500 nM α-thrombin. FIG. 2B shows the relative fluorescence of 6-FAM in a solution of mixed 100 nM FQ-15Ap and 100 nM α-thrombin, 200 nM AT3 (⋄), 500 nM HirF (▪) or 300 nM AHT (Δ) was added at 0 sec. and fluorescence of 6-FAM was continuously monitored.

FIG. 3 is a graph showing dual-labeled 27mer aptamer for α-thrombin/protein interactions. In a solution of mixed 100 nM FQ-27Ap and 100 nM α-thrombin, 300 nM AT3 (⋄), 500 nM HirF (▪) or 300 nM AHT (Δ) was added at 0 sec. and fluorescence of 6-FAM was continuously monitored.

FIGS. 4A and 4B are graphs showing TAMRA-labeled aptamers for α-thrombin/protein interactions based on fluorescence anisotropy. FIG. 4A shows TAMRA-labeled aptamers for α-thrombin/protein interactions in a solution of mixed 100 nM FQ-15Ap and 100 nM α-thrombin, 200 nM AT3 (⋄), 500 nM HirF (▪) or 300 nM AHT (Δ) was added at 0 sec. and anisotropy of TAMRA was recorded in real time. FIG. 4B shows the same experiments as in FIG. 4A using the T-27Ap aptamer. 200 nM AT3 (⋄), 500 nM HirF (▪) or 300 nM AHT (Δ) was added to the aptamer/α-thrombin mixture solution at 0 sec.

FIG. 5 is a gel showing binding between α-thrombin and anti-human thrombin (AHT) confirmed by gel electrophoresis on a 7.5% native Tris-HCl gel. Left lane contains 5 μL 10 μM α-thrombin. Middle lane had 1 μL 32 μM AHT. Right lane is a mixture of 1 μL 32 μM AHT and 5 μL 10 μM α-thrombin.

FIG. 6 is a graph showing the order of incubation with thrombin. 500 nM HirF was first incubated with 100 nM thrombin () and then 100 nM FQ-1 5Ap was added at time 0 to replace HirF. In another case (Δ), 100 nM FQ-15Ap was incubated with 100 nM thrombin first and 500 nM HirF was added later at time 0. Completion of reactions was monitored using fluorescence changes of 6-FAM.

FIGS. 7A -7B are graphs showing the time of completion of the reaction.

FIG. 7A: 100 nM T-15Ap and 100 nM thrombin were first incubated. Various amounts of AT3 were added at time 0: () 100 nM; (⋄) 200 nM; (▴) 300 nM. FIG. 7B: various concentrations of T-15Ap were incubated with 100 nM thrombin: () 50 nM; (⋄) 100 nM; (▴) 200 nM. Then 300 nM of AHT was added at time 0. Completion of reactions was monitored using anisotropy changes.

FIG. 8 is a schematic illustration showing light-controlled protein activity by azobenzene-modified aptamer.

FIG. 9 is a schematic illustration showing fiber-based controlled release of an aptamer drug in a patient.

FIG. 10 is a graph showing inhibition of thrombin by 15Ap and DA-8S monitored by measuring scattering light. ▪ for 15Ap and ▴ for DA-8S. Thrombin was added at near 500 second.

### DETAILED DESCRIPTION

The invention describes a label-free and versatile method for real-time protein interaction study comprising DNA aptamers. In particular, the invention is directed to use of protein-binding aptamers for label-free protein-protein interactions. The invention utilizes two signal transduction strategies, FRET measurement and fluorescence anisotropy, to monitor the binding events between the aptamer-binding protein—“bait protein”, and a second protein—“prey protein”.

Before the present invention is disclosed and described, it is to be understood that this invention is not limited to the particular structures, process steps, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

Definitions

In accordance with the present invention and as used herein, the following terms are defined with the following meanings, unless explicitly stated otherwise.

As used herein, “a”, “an,” and “the” include plural references unless the context clearly dictates otherwise.

The term “biomolecule” refers to DNA, RNA (including MRNA, rRNA, tRNA and tmRNA), nucleotides and nucleosides.

A base “position” as used herein refers to the location of a given base or nucleotide residue within a nucleic acid.

As used herein, the term “array” refers to an ordered spatial arrangement, particularly an arrangement of immobilized biomolecules.

As used herein, the term “addressable array” refers to an array wherein the individual elements have precisely defined x and y coordinates, so that a given element at a particular position in the array can be identified.

As used herein, the terms “probe” and “biomolecular probe” refer to a biomolecule used to detect a complementary biomolecule. Examples include antigens that detect antibodies, oligonucleotides that detect complimentary oligonucleotides, and ligands that detect receptors. Such probes are preferably immobilized on a microelectrode comprising a substrate.

As used herein, the terms “bioarray,” “biochip” and “biochip array” refer to an ordered spatial arrangement of immobilized biomolecules on a microelectrode arrayed on a solid supporting substrate. Preferred probe molecules include aptamers, nucleic acids, oligonucleotides, peptides, ligands, antibodies and antigens; peptides and proteins are the most preferred probe species. Biochips, as used in the art, encompass substrates containing arrays or microarrays, preferably ordered arrays and most preferably ordered, addressable arrays, of biological molecules that comprise one member of a biological binding pair. Typically, such arrays are oligonucleotide arrays comprising a nucleotide sequence that is complementary to at least one sequence that may be or is expected to be present in a biological sample. Alternatively, and preferably, proteins, peptides or other small molecules can be arrayed in such biochips for performing, inter alia, immunological analyses (wherein the arrayed molecules are antigens) or assaying biological receptors (wherein the arrayed molecules are ligands, agonists or antagonists of said receptors).

As used herein, the term “aptamer” or “selected nucleic acid binding species” shall include non-modified or chemically modified RNA or DNA. The method of selection may be by, but is not limited to, affinity chromatography and the method of amplification by reverse transcription (RT) or polymerase chain reaction (PCR).

As used herein, the term “signaling aptamer” shall include aptamers with reporter molecules, preferably a fluorescent dye, appended to a nucleotide in such a way that upon conformational changes resulting from the aptamer's interaction with a ligand, the reporter molecules yields a differential signal, preferably a change in fluorescence intensity.

As used herein, the terms “ligand,” “target,” and “bait” are used interchangeably throughout the specification and includes any molecule that binds to the aptamer.

As used herein, the term “fragment or segment”, as applied to a nucleic acid sequence, gene or polypeptide, will ordinarily be at least about 5 contiguous nucleic acid bases (for nucleic acid sequence or gene) or amino acids (for polypeptides), typically at least about 10 contiguous nucleic acid bases or amino acids, more typically at least about 20 contiguous nucleic acid bases or amino acids, usually at least about 30 contiguous nucleic acid bases or amino acids, preferably at least about 40 contiguous nucleic acid bases or amino acids, more preferably at least about 50 contiguous nucleic acid bases or amino acids, and even more preferably at least about 60 to 80 or more contiguous nucleic acid bases or amino acids in length. “Overlapping fragments” as used herein, refer to contiguous nucleic acid or peptide fragments which begin at the amino terminal end of a nucleic acid or protein and end at the carboxy terminal end of the nucleic acid or protein. Each nucleic acid or peptide fragment has at least about one contiguous nucleic acid or amino acid position in common with the next nucleic acid or peptide fragment, more preferably at least about three contiguous nucleic acid bases or amino acid positions in common, most preferably at least about ten contiguous nucleic acid bases amino acid positions in common.

“Biological samples” include solid and body fluid samples. The biological samples used in the present invention can include cells, protein or membrane extracts of cells, blood or biological fluids such as ascites fluid or brain fluid (e.g., cerebrospinal fluid). Examples of solid biological samples include, but are not limited to, samples taken from tissues of the central nervous system, bone, breast, kidney, cervix, endometrium, head/neck, gallbladder, parotid gland, prostate, pituitary gland, muscle, esophagus, stomach, small intestine, colon, liver, spleen, pancreas, thyroid, heart, lung, bladder, adipose, lymph node, uterus, ovary, adrenal gland, testes, tonsils and thymus. Examples of “body fluid samples” include, but are not limited to blood, serum, semen, prostate fluid, seminal fluid, urine, saliva, sputum, mucus, bone marrow, lymph, and tears.

“Sample” is used herein in its broadest sense. A sample comprising polynucleotides, polypeptides, peptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; and the like.

“Marker” in the context of the present invention refers to a polypeptide (of a particular apparent molecular weight) which is differentially present in a sample taken from patients having a disease or disorder as compared to a comparable sample taken from control subjects (e.g., a person with a negative diagnosis, normal or healthy subject).

“Complementary” in the context of the present invention refers to detection of at least two biomarkers, which when detected together provides increased sensitivity and specificity as compared to detection of one biomarker alone.

The phrase “differentially present” refers to differences in the quantity and/or the frequency of a marker present in a sample taken from patients having for example, cancer as compared to a control subject. For example, a marker can be a polypeptide which is present at an elevated level or at a decreased level in samples of patients with cancer (e.g. tumor antigen) compared to samples of control subjects. Alternatively, a marker can be a polypeptide which is detected at a higher frequency or at a lower frequency in samples of patients compared to samples of control subjects. A marker can be differentially present in terms of quantity, frequency or both.

A polypeptide is differentially present between the two samples if the amount of the polypeptide in one sample is statistically significantly different from the amount of the polypeptide in the other sample. For example, a polypeptide is differentially present between the two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively or additionally, a polypeptide is differentially present between the two sets of samples if the frequency of detecting the polypeptide in samples of patients' suffering from disease or any disorder, is statistically significantly higher or lower than in the control samples. For example, a polypeptide is differentially present between the two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

“Diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

A “test amount” of a marker refers to an amount of a marker present in a sample being tested. A test amount can be either in absolute amount (e.g., μg/ml) or a relative amount (e.g., relative intensity of signals).

A “diagnostic amount” of a marker refers to an amount of a marker in a subject's sample that is consistent with a diagnosis of disease or any other disorder. A diagnostic amount can be either in absolute amount (e.g., μg/ml) or a relative amount (e.g., relative intensity of signals).

A “control amount” of a marker can be any amount or a range of amount which is to be compared against a test amount of a marker. For example, a control amount of a marker can be the amount of a marker in a person without disease or any other disorder. A control amount can be either in absolute amount (e.g., μg/ml) or a relative amount (e.g., relative intensity of signals).

“Probe” refers to a device that is removably insertable into a gas phase ion spectrometer and comprises a substrate having a surface for presenting a marker for detection. A probe can comprise a single substrate or a plurality of substrates.

“Substrate” or “probe substrate” refers to a solid phase onto which an adsorbent can be provided (e.g., by attachment, deposition, etc.).

“Adsorbent” refers to any material capable of adsorbing a marker. The term “adsorbent” is used herein to refer both to a single material (“monoplex adsorbent”) (e.g., a compound or functional group) to which the marker is exposed, and to a plurality of different materials (“multiplex adsorbent”) to which the marker is exposed. The adsorbent materials in a multiplex adsorbent are referred to as “adsorbent species.” For example, an addressable location on a probe substrate can comprise a multiplex adsorbent characterized by many different adsorbent species (e.g., anion exchange materials, metal chelators, or antibodies), having different binding characteristics. Substrate material itself can also contribute to adsorbing a marker and may be considered part of an “adsorbent.”

“Adsorption” or “retention” refers to the detectable binding between an absorbent and a marker either before or after washing with an eluant (selectivity threshold modifier) or a washing solution.

“Eluant” or “washing solution” refers to an agent that can be used to mediate adsorption of a marker to an adsorbent. Eluants and washing solutions are also referred to as “selectivity threshold modifiers.” Eluants and washing solutions can be used to wash and remove unbound materials from the probe substrate surface.

“Resolve,” “resolution,” or “resolution of marker” refers to the detection of at least one marker in a sample. Resolution includes the detection of a plurality of markers in a sample by separation and subsequent differential detection. Resolution does not require the complete separation of one or more markers from all other biomolecules in a mixture. Rather, any separation that allows the distinction between at least one marker and other biomolecules suffices.

“Gas phase ion spectrometer” refers to an apparatus that measures a parameter which can be translated into mass-to-charge ratios of ions formed when a sample is volatilized and ionized. Generally ions of interest bear a single charge, and mass-to-charge ratios are often simply referred to as mass. Gas phase ion spectrometers include, for example, mass spectrometers, ion mobility spectrometers, and total ion current measuring devices.

“Mass spectrometer” refers to a gas phase ion spectrometer that includes an inlet system, an ionization source, an ion optic assembly, a mass analyzer, and a detector.

“Laser desorption mass spectrometer” refers to a mass spectrometer which uses laser as means to desorb, volatilize, and ionize an analyte.

“Detect” refers to identifying the presence, absence or amount of the object to be detected.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

Aptamers

Aptamer polynucleotides are typically single-stranded standard phosphodiester DNA (ssDNA). Close DNA analogs can also be incorporated into the aptamer as described below.

A typical aptamer discovery procedure is described below:

A polynucleotide comprising a randomized sequence between “arms” having constant sequence is synthesized. The arms can include restriction sites for convenient cloning and can also function as priming sites for PCR primers. The synthesis can easily be performed on commercial instruments.

The target protein is treated with the randomized polynucleotide. The target protein can be in solution and then the complexes immobilized and separated from unbound nucleic acids by use of an antibody affinity column. Alternatively, the target protein might be immobilized before treatment with the randomized polynucleotide.

The target protein-polynucleotide complexes are separated from the uncomplexed material and then the bound polynucleotides are separated from the target protein. The bound nucleic acid can then be characterized, but is more commonly amplified, e.g. by PCR and the binding, separation and amplification steps are repeated. In many instances, use of conditions increasingly promoting separation of the nucleic acid from the target protein, e.g. higher salt concentration, in the binding buffer used in step 2) in subsequent iterations, results in identification of polynucleotides having increasingly high affinity for the target protein.

The nucleic acids showing high affinity for the target proteins are isolated and characterized. This is typically accomplished by cloning the nucleic acids using restriction sites incorporated into the arms, and then sequencing the cloned nucleic acid.

The affinity of aptamers for their target proteins is typically in the nanomolar range, but can be as low as the picomolar range. That is K_{D} is typically 1 pM to 500 nM, more typically from 1 pM to 100 nM. Apatmers having an affinity of K_{D} in the range of 1 pM to 10 nM are also useful.

Aptamer polynucleotides can be synthesized on a commercially available nucleic acid synthesizer by methods known in the art. The product can be purified by size selection or chromatographic methods.

Aptamer polynucleotides are typically from about 10 to 200 nucleotides long, more typically from about 10 to 100 nucleotides long, still more typically from about 10 to 50 nucleotides long and yet more typically from about 10 to 25 nucleotides long. A preferred range of length is from about 10 to 50 nucleotides.

The aptamer sequences can be chosen as a desired sequence, or random or partially random populations of sequences can be made and then selected for specific binding to a desired target protein by assay in vitro. Any of the typical nucleic acid-protein binding assays known in the art can be used, e.g. “Southwestern” blotting using either labeled oligonucleotide or labeled protein as the probe. See also U.S. Pat. No. 5,445,935 for a fluorescence polarization assay of protein-nucleic acid interaction.

Appropriate nucleotides for aptamer synthesis and their use, and reagents for covalent linkage of proteins to nucleic acids and their use, are considered known in the art.

A desired aptamer-protein complex, for example, aptamer-thrombin complex of the invention can be labeled and used as a diagnostic agent in vitro in much the same manner as any specific protein-binding agent, e.g. a monoclonal antibody. Thus, an aptamer-protein complex of the invention can be used to detect and quantitate the amount of its target protein in a sample, e.g. a blood sample, to provide diagnosis of a disease state correlated with the amount of the protein in the sample.

A desired aptamer-target/bait molecular complex can also be used for diagnostic imaging. In imaging uses, the complexes are labeled so that they can be detected outside the body. Typical labels are radioisotopes, usually ones with short half-lives. The usual imaging radioisotopes, such as ¹²³I, ¹²⁴I, ¹²⁵I, ¹³¹I, ^{99m}TC, ¹⁸⁶Re, ¹⁸⁸Re, ⁶⁴Cu, ⁶⁷Cu, ²¹²Bi, ²¹³Bi, ⁶⁷Ga, ⁹⁰Y, ¹¹¹In, ¹⁸F, ³H, ¹⁴C, 35S or ³²P can be used. Nuclear magnetic resonance (NMR) imaging enhancers, such as gadolinium-153, can also be used to label the complex for detection by NMR. Methods and reagents for performing the labeling, either in the polynucleotide or in the protein moiety, are considered known in the art.

Aptamer Selection

Aptamers configured to bind to specific target molecules can be selected, e.g., by synthesizing an initial heterogeneous population of oligonucleotides, and then selecting oligonucleotides within the population that bind tightly to a particular target molecule. Once an aptamer that binds to a particular target molecule has been identified, it can be replicated using a variety of techniques known in biological and other arts, e.g., by cloning and polymerase chain reaction (PCR) amplification followed by transcription.

The synthesis of a heterogeneous population of oligonucleotides and the selection of aptamers within that population can be accomplished using a procedure known as the Systematic Evolution of Ligands by Exponential Enrichment or SELEX. The SELEX method is described in, e.g., Gold et al., U.S. Pat. Nos. 5,270,163 and 5,567,588; Fitzwater et al., “A SELEX Primer,” Methods in Enzymology, 267:275-301 (1996); and in Ellington and Szostak, “In Vitro Selection of RNA Molecules that Bind Specific Ligands,” Nature, 346:818-22. Briefly, a heterogeneous DNA oligomer population is synthesized to provide candidate oligomers for the in vitro selection of aptamers. This initial DNA oligomer population is a set of random sequences 15 to 100 nucleotides in length flanked by fixed 5′ and 3′ sequences 10 to 50 nucleotides in length. The fixed regions provide sites for PCR primer hybridization and, in one implementation, for initiation of transcription by an RNA polymerase to produce a population of RNA oligomers. The fixed regions also contain restriction sites for cloning selected aptamers. Many examples of fixed regions can be used in aptamer evolution. See, e.g., Conrad et al., “In Vitro Selection of Nucleic Acid Aptamers That Bind Proteins,” Methods in Enzymology, 267:336-83 (1996); Ciesiolka et al., “Affinity Selection-Amplification from Randomized Ribooligonucleotide Pools,” Methods in Enzymology, 267:315-35 (1996); Fitzwater, supra.

Aptamers are selected in a 5 to 100 cycle procedure. In each cycle, oligomers are bound to the target molecule, purified by isolating the target to which they are bound, released from the target, and then replicated by 20 to 30 generations of PCR amplification.

Aptamer selection is similar to evolutionary selection of a function in biology. Subjecting the heterogeneous oligonucleotide population to the aptamer selection procedure described above is analogous to subjecting a continuously reproducing biological population to 10 to 20 severe selection events for the function, with each selection separated by 20 to 30 generations of replication.

Modified Aptamers

Heterogeneity is introduced, e.g., at the beginning of the aptamer selection procedure, and does not occur throughout the replication process. Alternatively, heterogeneity can be introduced at later stages of the aptamer selection procedure.

Various oligomers can be used for aptamer selection, including, e.g., 2′-fluoro-ribonucleotide oligomers, NH₂-substituted and OCH₃-substituted ribose aptamers, and deoxyribose aptamers. RNA and DNA populations are equally capable of providing aptamers configured to bind to any type of target molecule. Within either population, the selected aptamers occur at a frequency of 10⁹ to 10^{13,} see Gold et al., “Diversity of Oligonucleotide Functions,” Annual Review of Biochemistry, 64:763-97 (1995).

Using 2′-fluoro-ribonucleotide oligomers is likely to increase binding affinities ten to one hundred fold over those obtained with unsubstituted ribo- or deoxyribo-oligonucleotides. See Pagratis et al., “Potent 2′-amino and 2′ fluoro 2′deoxyribonucleotide RNA inhibitors of keratinocyte growth factor” Nature Biotechnology, 15:68-73. Such modified bases provide additional binding interactions and increase the stability of aptamer secondary structures. These modifications also make the aptamers resistant to nucleases, a significant advantage for real world applications of the system. See Lin et al., “Modified RNA sequence pools for in vitro selection” Nucleic Acids Research, 22:5229-34 (1994); Pagratis, supra.

Modified aptamers are aptamers having at least two types of nucleotides, such as both deoxyribonucleotides and ribonucleotides, ribonucleotides and modified nucleotides, or two different types of modified nucleotides. One form of an aptamer is peptide nucleic acid/nucleic acid aptamer (PNA/NAP). For example, 5′-PNA-DNA-3′ or 5′-PNA-RNA-3′ aptamers may be used. The DNA and RNA portions of such aptamers can have random or degenerate sequences. Other forms of aptamers are, for example, 5′-(2′-O-Methyl)RNA-RNA-3′ or 5′-(2′-O-Methyl)RNA-DNA-3′.

Many modified nucleotides (nucleotide analogs) are known and can be used in aptamer synthesis. A nucleotide analog is a nucleotide which contains some type of modification to either the base, sugar, or phosphate moieties. Modifications to the base moiety would include natural and synthetic modifications of A, C, G. and T/U as well as different purine or pyrimidine bases, such as uracil-5-yl, hypoxanthin-9-yl (I), and 2-aminoadenin-9-yl. A modified base includes but is not limited to locked nucleic acids (LNA), 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Atigewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation.

Nucleotide analogs can also include modifications of the sugar moiety. Modifications to the sugar moiety would include natural modifications of the ribose and deoxyribose as well as synthetic modifications. Sugar modifications include but are not limited to the following modifications at the 2′ position: OH; F; O—, S—, or N-alkyl; O—, S—, or N-alkenyl; O— S— or N-alkynyl; or O-alkyl-O-alkyl, wherein the alkyl, alkenyl and alkynyl may be substituted or unsubstituted C1 to C10, alkyl or C2 to C10 alkenyl and alkynyl. 2′ sugar modifications also include but are not limited to —O[(CH₂)ₙO]m CH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m are from 1 to about 10.

Other modifications at the 2′ position include but are not limited to: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aninoalkylphosphoramidates, thionophosphoramidates, thionoalkylphosphonates, thionoalkylphosphotriesters, and boranophosphates. It is understood that these phosphate or modified phosphate linkages between two nucleotides can be through a 3′-5′ linkage or a 2′-5′ linkage, and the linkage can comprise inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

It is understood that nucleotide analogs need only comprise a single modification, but may also comprise multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize and hybridize to complementary nucleic acids in a Watson-Crick or Hoögsteen manner, but which are linked together through a moiety other than a phosphate moiety. Nucleotide substitutes are able to conform to a double helix type structure when interacting with the appropriate target nucleic acid. Substitutes for the phosphate can be for example, short chain alkyl or cycloalkyl internucleoside linkages, mixed heteroatom and alkyl or cycloalkyl intemucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thiofonnacetyl backbones; methylene fonnacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. (See also Nielsen et al., Science 254:1497-1500 (1991)).

Aptamers can comprise nucleotides and can be made up of different types of nucleotides or the same type of nucleotides. For example, one or more of the nucleotides can be ribonucleotides, 2′-O-methyl ribonucleotides, or a mixture of ribonucleotides and 2′-O-methyl ribonucleotides; about 10% to about 50% of the nucleotides can be ribonucleotides, 2′-O-methyl ribonucleotides, or a mixture of ribonucleotides and 2′-O-methyl ribonucleotides; about 50% or more of the nucleotides can be ribonucleotides, 2′-O-methyl ribonucleotides, or a mixture of ribonucleotides and 2′-O-methyl ribonucleotides; or all of the nucleotides are ribonucleotides, 2′-O-methyl ribonucleotides, or a mixture of ribonucleotides and 2′-O-methyl ribonucleotides. The nucleotides can comprise bases (that is, the base portion of the nucleotide) and can (and normally will) comprise different types of bases.

Detection of Target Molecules

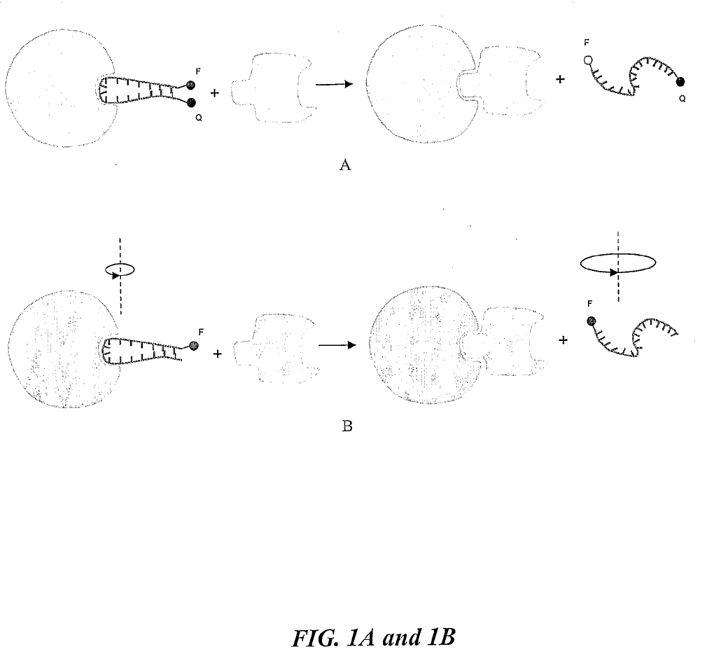

In a preferred embodiment, the invention provides a direct use of protein-binding aptamers for label-free protein-protein interactions. Preferably, two signal transduction strategies, FRET measurement and fluorescence anisotropy, are used to monitor the binding events between the aptamer-binding protein—“bait protein”, and a second protein—“prey protein”. A schematic representation of the invention is provided which is not meant to limit or construe the invention in any way. As illustrated in FIG. 1A, an aptamer can be labeled with a fluorophore and a quencher to have internal FRET. Binding of the aptamer to the bait protein causes a quenched fluorescence, while the binding of the prey protein to the bait protein may either displace the aptamer and result in a restoration of fluorescence (sequential incubation) or inhibit the binding of the aptamer and prevent quenching (co-incubation). In another approach, illustrated in FIG. 1B, an aptamer is labeled with only one fluorophore and the fluorescence anisotropy of the aptamer or the aptamer complex is monitored in real time. Binding of the aptamer to a much larger bait protein molecule will result in increased fluorescence anisotropy. Further change in the anisotropy of the aptamer can be triggered by the interaction between the bait and prey proteins. Co-binding of aptamer and a protein on the same target protein is also possible to be monitored in this way. Neither approach requires labeling of the interacting target protein or the probe protein, allowing true real-time monitoring of the interactions between the two proteins based on their unaffected biological activities. While each one excels in different aspects of protein interaction study, the combination of the two fluorescence techniques was found to be capable of providing detailed knowledge about the kinetics of the protein-protein binding as well as the mechanism and binding site information of the interactions. This is not possible with other current techniques.

In another preferred embodiment, the invention provides for aptamer labeling techniques. Methods for the molecular aptamers's application have been developed for detecting biomarkers, small molecules and drugs and for protein interactions. Aptamers have been developed as molecular probes for easy and effective detection of small drug molecules, proteins and inter-molecular interactions. Compared with previous techniques, the methods disclosed herein, have enabled aptamers to have high sensitivity and extremely high selectivity for their targets. Aptamers are highly stable and can be easily modified for different signal transduction mechanisms.

Aptamers are nucleic acid oligonucleotides that may be selected using a systematic evolution of ligands via an exponential enrichment (SELEX) process (Tuerk, C. & Gold L. (1990) Science 249, 505-510; Ellington, A. D. & Szostak, J. W. (1990) Nature 346, 818-822). Compared to antibodies, aptamers can have similar affinity to their protein targets but are much smaller and much easier to produce. Quite tolerant to external environment changes and internal modifications, aptamers can be conveniently labeled for various applications.

In another preferred embodiment, the invention provides unique biolabeling methods and bioanalytical techniques that can use aptamers efficiently for disease-related protein, small molecules and biomarkers and for high throughput protein-protein interaction studies.

In another preferred embodiment, aptamers as disease markers are developed for drug related small molecules and for bioanalysis. Aptamers of the invention are useful in areas such as disease diagnosis and therapeutics, small molecule detection, drug discovery and in biomedical and biotechnological studies.

In another preferred embodiment, the invention provides aptamers for protein-protein interaction and detection. Preferably, aptamers are systematically selected nucleic acids that have high affinity and selectivity for their target proteins. The methods described herein, enable label-free analysis of proteins in real time and homogeneous solutions based on aptamers. Fluorescence steady state and polarization measurements are used for signal transduction. For example, in steady state, the aptamer is labeled with two fluorophores that have overlapping excitation and emission spectra. When bound to the target protein, the binding-induced conformational change of the aptamer causes two fluorophores to be in close proximity and change their fluorescence intensity because of fluorescence resonance energy transfer (FRET). This process can be used to report the presence of the target protein without labeling it. By looking at the fluorescence of either one of the two fluorophores, a fluorescence quenching assay or a fluorescence generating assay depending on the specific application is used, as described in detail in the Examples which follow. In the polarization measurements, the aptamer is labeled with only one fluorophore. Binding to a much larger protein target results in a slower diffusional rotation of the aptamer and increased fluorescence anisotropy of the fluorophore. The methods have demonstrated highly sensitive protein detection with excellent selectivity in real time using both FRET and anisotropy approaches.

In another preferred embodiment, the invention provides aptamers and methods to determine protein functions. For example, in a competitive assay, the aptamer/target protein binding complex can be disrupted by a third molecule, either a protein or a drug molecule, if the third molecule can interact with the target protein. This protein-molecule interaction can be readily reflected in real time by changes in the fluorescent signals of the aptamer. By analyzing FRET or anisotropy of the dye-labeled aptamer, protein-protein and protein-small molecule interactions in homogeneous solution have been monitored and information about the kinetics and binding sites of the interactions have been gathered as described in the Examples which follow. All this can be done easily and quickly without labeling the protein or the third molecule, which gives the most “true-to-life” insight into protein functions based on the unaffected protein structures.

In another preferred embodiment, the invention provides for aptamer based assays in determining protein and drug molecule interactions. Fluorescence assays developed herein, for protein analysis and protein function study are easily adapted to large-scale formats, such as a 96-well array, for high-throughput protein study. Diagnoses of diseases including cancers can be carried out by identify certain protein markers that are present in the cells. By developing aptamers for different cancer marker proteins, bioarrays that have the capability of sensitive multiplex cancer marker detection can be developed. Based on the aptamer assay, the analysis of the cell content is highly efficient. The fluorescence signals obtained from the array generate a pattern which shows the presence of different cancer related proteins. By comparing the patterns acquired from different cell samples, cancer diagnoses can be accomplished with great ease and accuracy.

In another preferred embodiment, the invention provides for the dequenching of fluorophores bound to aptamers used in real time protein detection. Protein-binding aptamer based assays have been shown, herein, to be capable of sensitive protein detection in real time. Preferably, the signal transduction mechanisms used in such detection include fluorescence resonance energy transfer (FRET) and fluorescence anisotropy (FA). For example, in FRET, a fluorophore and a quencher are labeled on the aptamer and a protein-binding induced conformational change of the aptamer is required to trigger a fluorescence signal change. In FA, two polarizers are needed which causes much lower detected fluorescence intensity compared to steady state measurements. A small dynamic range is usually another problem with FA for sensitive protein detection. As described in the examples which follow, some fluorophores, when attached to certain positions on the aptamer, could display a significant fluorescence enhancement upon protein binding. Without wishing to be bound by theory, this result may be due to the quenching of the fluorophore by the nucleic acid bases of the aptamer. Binding to the protein can alleviate the fluorophore from the quenching environments and cause a restored fluorescence. This new finding has allowed us to construct an assay that requires only one dye on the aptamer, as in FA, while having similar sensitivity and dynamic range as in FRET. It may also reduce the concern of aptamer conformational change that is necessary with FRET. The dequenching of fluorophores on aptamers (DFA) assay provides an economical and sensitive alternative for real-time protein detection in homogeneous solutions.

Fluorescence resonance energy transfer (FRET) occurs between the electronic excited states of two fluorophores when they are in sufficient proximity to each other, in which the excited-state energy of the donor fluorophore is transferred to the acceptor fluorophore. The result is a decrease in the lifetime and a quenching of fluorescence of the donor species and a concomitant increase in the fluorescence intensity of the acceptor species. In one application of this principle, a fluorescent moiety is caused to be in close proximity to a quencher molecule. Donor and acceptor molecules operate in a set wherein one or more acceptor molecules accepts energy from one or more donor molecules, or otherwise quenches signal from the donor molecule, when the donor and acceptor molecules are closely associated. In one embodiment, the donor and acceptor molecules are about 30 to about 200 Å apart or about 10 to about 40 nucleotides apart. Transfer of energy may occur through collision of the closely associated molecules of a set, or through a non-radiative process such as fluorescence resonance energy transfer (FRET). For FRET to occur, transfer of energy between donor and acceptor molecules requires that the molecules be close in space and that the emission spectrum of a donor have substantial overlap with the absorption spectrum of the acceptor (Yaron et al. Analytical Biochemistry, 95, 228-235 (1979), the teachings of which are incorporated herein by reference). Alternatively, intramolecular energy transfer may occur between very closely associated donor and acceptor molecules (e.g., within 10 Å) whether or not the emission spectrum of a donor molecule has a substantial overlap with the absorption spectrum of the acceptor molecule (Yaron et al.) This process is referred to as intramolecular collision since it is believed that quenching is caused by the direct contact of the donor and acceptor molecule (Yaron et al.).

Because the efficiency of both collision and non-radiative transfer of energy between the donor and acceptor molecules is directly dependent on the proximity of the donor and acceptor molecules, formation and dissociation of the complexes of this invention can be monitored by measuring at least one physical property of at least one member of the set which is detectably different when the complex is formed, as compared with when the aptamers and target/bait exist independently and unassociated. Preferably, the means of detection will involve measuring fluorescence of an acceptor fluorophore of a set or the fluorescence of the donor fluorophore in a set containing a fluorophore and quencher pair (e.g. a donor and acceptor). While not wishing to be bound by theory, the fluorescent molecules may interact with one another via hydrophobic interactions, thereby reducing the adverse effect of distance between the donor and acceptor fluorescent molecules. Thus, fluorescence energy transfer can occur when the donor and acceptor fluorescent molecules are up to about 40 nucleotides away from each other.

In one embodiment of the present invention, the 3′-end of the aptamer is labeled with N, N₁, N, N₁-tetramethyl-6-carboxy rhodamine (TAMRA). Donor and acceptor molecules suitable for FRET are well known in the art (see page 46 of R. P. Haugland, Handbook of Fluorescent Probes and Research Chemicals, 6th ed.; Molecular Probes, Oregon, the teachings of which are incorporated herein by reference). Typically, to obtain fluorescence resonance energy transfer, the donor fluorescent molecule has a shorter excitation wavelength than the acceptor fluorescent molecule and the donor emission wavelength overlaps with the acceptor excitation wavelength, to allow transfer of energy from the donor to the acceptor. Preferred fluorophores are fluorescein and derivatives thereof, such as 5-(2′-aminoethyl)-aminoapthalene-1-sulfonic acid (EDANS) and rhodamine and derivatives thereof such as Cy3, Cy5 and Texas Red. Suitable donor/acceptor pairs are, for example, fluorescein/tetramethyrhodamine, IAEDANS/fluorescein and EDANS/DABCYL. In another embodiment of the present invention, the same fluorescent molecule is used for the donor and acceptor. In this embodiment, the wavelength used to excite the detection complexes is polarized. Unpolarized emission detected is indicative of FRET. In this embodiment, it is preferable to remove unincorporated labeled nucleotides (e.g., by washing) to improve the detection signal.

Those of ordinary skill in the art will recognize that labeled, unlabeled and modified nucleotides are readily available for the method of the present invention. They can be synthesized using commercially available instrumentation and reagents or they can be purchased from numerous commercial vendors of custom manufactured oligonucleotides.

Aptamers have great potential in molecular recognition due to their excellent structural stability and exceptional flexibility with various intra-molecular modifications. While previous work has been focused on using aptamers as probes for direct detection of their target molecules, this invention describes novel applications for aptamers in areas where understanding of the interactions between known proteins and other molecules bears great significance.

In another preferred embodiment, aptamers are labeled with a fluorophore and a quencher to form intra-molecular FRET. Preferably, the folded conformations of the aptamers are stabilized by binding to their target molecules and produce a fluorescence signal change of the fluorophore induced by FRET when the aptamer binds to its target. Preferably, the target-binding induced FRET cause between about 40% up to 100% fluorescence quenching.

In another preferred embodiment, FRET can be formed within an aptamer even if the aptamer lacks the necessary conformational changes accompanying the binding to the target molecules.

In another preferred embodiment, the invention provides a fluorescence anisotropy method which relies on the relatively smaller sizes of aptamers compared to proteins. It is demonstrated herein that aptamer based anisotropy probes can provide sufficient signal change for protein-protein interaction study.

Solid-State Detectors

Solid-state detectors are solid-state substrates or supports to which aptamers or detection molecules have been coupled. A preferred form of solid-state detector is an array detector. An array detector is a solid-state detector to which multiple different aptamers or detection molecules have been coupled in an array, grid, or other organized pattern.

Solid-state substrates for use in solid-state detectors can include any solid material to which oligonucleotides can be coupled. This includes materials such as acrylamide, cellulose, nitrocellulose, glass, gold, polystyrene, polyethylene vinyl acetate, polypropylene, polymethacrylate, polyethylene, polyethylene oxide, glass, polysilicates, polycarbonates, polytetrafluoroethylene (TEFLON™), fluorocarbons, nylon, silicon rubber, polyanhydrides, polyglycolic acid, polylactic acid, polyorthoesters, functionalized silane, polypropylfumerate, collagen, glycosaminoglycans, and polyamino acids. Solid-state substrates can have any useful form including thin films or membranes, beads, bottles, dishes, fibers, optical fibers, woven fibers, chips, compact disks, shaped polymers, particles and microparticles. A chip is a rectangular or square small piece of material. Preferred forms for solid-state substrates are thin films, beads, or chips.

Aptamers immobilized on a solid-state substrate allow capture of the products of the disclosed amplification method on a solid-state detector. Such capture provides a convenient means of washing away reaction components that might interfere with subsequent detection steps. By attaching different aptamers to different regions of a solid-state detector, different target/bait can be captured at different, and therefore diagnostic, locations on the solid-state detector. For example, in a multiplex assay, aptamers specific for numerous different targets (each representing a different target sequence amplified via a different set of primers) can be immobilized in an array, each in a different location. Capture and detection will occur only at those array locations corresponding to aptamers for which the corresponding target sequences were present in a sample.

Methods for immobilization of oligonucleotides to solid-state substrates are well established. Oligonucleotides, including aptamers and detection probes, can be coupled to substrates using established coupling methods. For example, suitable attachment methods are described by Pease et al., Proc. Natl. Acad. Sci. USA 91(11):5022-5026 (1994), and Khrapko et al., Mol Biol. (Mosk) (USSR) 25:718-730 (1991). A method for immobilization of 3′-amine oligonucleotides on casein-coated slides is described by Stimpson et al., Proc. Natl. Acad. Sci. USA 92:6379-6383 (1995). A preferred method of attaching oligonucleotides to solid-state substrates is described by Guo et al., Nucleic Acids Res. 22:5456-5465 (1994). Examples of nucleic acid chips and arrays, including methods of making and using such chips and arrays, are described in U.S. Pat. Nos. 6,287,768, 6,288,220, 6,287,776, 6,297,006, and 6,291,193 which are hereby incorporated by reference in their entirety.

Detection Labels

To aid in detection and quantitation of molecules using the disclosed methods (see the Examples which follow), detection labels can be directly incorporated into aptamers or can be coupled to detection molecules. As used herein, a detection label is any molecule that can be associated with aptamers, directly or indirectly, and which results in a measurable, detectable signal, either directly or indirectly. A label may be any moiety covalently attached to an oligonucleotide or nucleic acid analog. Many such labels for incorporation into nucleic acids or coupling to nucleic acid probes are known to those of skill in the art. Examples of detection labels suitable for use in the disclosed method are radioactive isotopes, fluorescent molecules, phosphorescent molecules, enzymes, antibodies, and ligands.

A preferred class of labels are detection labels, which may provide a signal for detection of the labeled oligonucleotide by fluorescence, chemiluminescence, and electrochemical luminescence. Fluorescent dyes useful for labeling oligonucleotides include fluoresceins, rhodamines, cyanines, and metal porphyrin complexes. Preferred fluorescein dyes include 6-carboxyfluorescein (6-FAM), 2′, 4′,1,4-tetrachlorofluorescein (TET), 2′,4′,5′,7′,1,4-hexachlorofluorescein (HEX), 2′,7′-dimethoxy-4′,5′-dichloro-6-carboxyrhodamin (JOE), 2′-chloro-5′-fluoro-7′,8′-fused phenyl-1,4-dichloro-6-caroxyflurescein (NED), 2′-chloro-7′-phenyl-1,4-dichloro-6-carboxyfluorescein (VIC), and (JODA). The 5-carboxyl, and other regio-isomers, may also have useful detection properties. Fluorescein and rhodamine dyes with 1,4-dichloro substituents are especially preferred.

Another preferred class of labels include quencher moieties. The emission spectra of a quencher moiety overlaps with a proximal intramolecular or intermolecular fluorescent dye such that the fluorescence of the fluorescent dye is substantially diminished, or quenched, by fluorescence resonance energy transfer (FRET). Oligonucleotides which are intramolecularly labeled with both fluorescent dye and quencher moieties are useful in nucleic acid hybridization assays, e.g. the “Taqman™” exonuclease-cleavage PCR assay.

Particularly preferred quenchers include but are not limited to (i) rhodamine dyes selected from the group consisting of tetramethyl-6-carboxyrhodamine (TAMRA), tetrapropano-6-carboxyrhodamine (ROX), and (ii) DABSYL, DABCYL, cyanine dyes including nitrothiazole blue (NTB), anthraquinone, malachite green, nitrothiazole, and nitroimidazole compounds and the like.

Preferred fluorescent labels are fluorescein (5-carboxyfluorescein-N-hydroxysuccinimide ester), rhodamine (5,6-tetramethyl rhodamine), and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. Other examples of fluorescein dyes include 6-carboxyfluorescein (6-FAM), 2′,4′,1,4,-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′, 8′-fused phenyl-1,4-dichloro-6-carboxyfluorescein (NED), and 2′-chloro-7′-phenyl-1,4-dichloro-6-carboxyfluorescein (VIC). Fluorescent labels can be obtained from a variety of commercial sources, including Amersham Pharmacia Biotech, Piscataway, N.J.; Molecular Probes, Eugene, Oreg.; and Research Organics, Cleveland, Ohio.

Examples of other suitable fluorescent labels include fluorescein isothiocyanate (FITC), 5,6-carboxymethyl fluorescein, Texas red, nitrobenz-2-oxa-1,3-diazol-4-yl (NBD), coumarin, dansyl chloride, rhodamine, amino-methyl coumarin (AMCA), Eosin, Erythrosin, BODIPY™, Cascade Blue™, Oregon Green™, pyrene, lissamine, xanthenes, acridines, oxazines, phycoerythrin, macrocyclic chelates of lanthanide ions such as quantum dye™, fluorescent energy transfer dyes, such as thiazole orange-ethidium heterodimer, and the cyanine dyes Cy3, Cy3.5, Cy5, Cy5.5 and Cy7. Examples of other specific fluorescent labels include 3-Hydroxypyrene 5,8,10-Tri Sulfonic acid, 5-Hydroxy Tryptamine (5-HT), Acid Fuchsin, Alizarin Complexon, Alizarin Red, Allophycocyanin, Aminocoumarin, Anthroyl Stearate, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, Blancophor FFG Solution, Blancophor SV, Bodipy F1, Brilliant Sulphoflavin FF, Calcien Blue, Calcium Green, Calcofluor RW Solution, Calcofluor White, Calcophor White ABT Solution, Calcophor White Standard Solution, Carbostyryl, Cascade Yellow, Catecholamine, Chinacrine, Coriphosphine O, Coumarin-Phalloidin, CY3.18, CY5.18, CY7, Dans (1-Dimethyl Amino Naphaline 5 Sulphonic Acid), Dansa (Diamino Naphtyl Sulphonic Acid), Dansyl NH—CH3, Diamino Phenyl Oxydiazole (DAO), Dimethylamino-5-Sulphonic acid, Dipyrrometheneboron Difluoride, Diphenyl Brilliant Flavine 7GFF, Dopamine, Erythrosin ITC, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow 5GF, Gloxalic Acid, Granular Blue, Haematoporphyrin, Indo-1, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nitrobenzoxadidole, Noradrenaline, Nuclear Fast Red, Nuclear Yellow, Nylosan Brilliant Flavin E8G, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Polyazaindacene Pontochrome Blue Black, Porphyrin, Primuline, Procion Yellow, Pyronine, Pyronine B, Pyrozal Brilliant Flavin 7GF, Quinacrine Mustard, Rhodamine 123, Rhodamine 5 GLD, Rhodamine 6G, Rhodamine B, Rhodarmine B 200, Rhodamine B Extra, Rhodamine BB, Rhodamine BG, Rhodamine WT, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulpho Rhodanine B Can C, Sulpho Rhodamine G Extra, Tetracycline, Thiazine Red R, Thioflavin S, Thioflavin TCN, Thioflavin 5, Thiolyte, Thiozol Orange, Tinopol CBS, True Blue, Ultralite, Uranine B, Uvitex SFC, Xylene Orange, and XRITC.

Additional labels of interest include those that provide for signal only when the aptamer with which they are associated is specifically bound to a target molecule, where such labels include: “molecular beacons” as described in Tyagi & Kramer, Nature Biotechnology (1996) 14:303 and EP 0 070 685 B1. Other labels of interest include those described in U.S. Pat. No. 5,563,037; WO 97/17471 and WO 97/17076.

Labeled nucleotides are a preferred form of detection label since they can be directly incorporated into the amplification products during synthesis. Examples of detection labels that can be incorporated into amplified nucleic acids include nucleotide analogs such as BrdUrd (5-bromodeoxyuridine, Hoy and Schimke, Mutation Research 290:217-230 (1993)), aminoallyldeoxyuridine (Henegariu et al., Nature Biotechnology 18:345-348 (2000)), 5-methylcytosine (Sano et al., Biochim. Biophys. Acta 951:157-165 (1988)), bromouridine (Wansick et al., J. Cell Biology 122:283-293 (1993)) and nucleotides modified with biotin (Langer et al., Proc. Natl. Acad. Sci. USA 78:6633 (1981)) or with suitable haptens such as digoxygenin (Kerkhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res., 22:3226-3232 (1994)).

Methods for detecting and measuring signals generated by detection labels are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary detection label coupled to the antibody. As used herein, detection molecules are molecules which interact with amplified nucleic acid and to which one or more detection labels are coupled.

The above-mentioned methods are superior to any method in the art allowing real time monitoring of protein-protein interactions without any modifications to the interacting proteins. The invention described herein, has many advantages over the prior art. There are many difficulties in determining the binding affinities of molecules: more target protein is necessary to cause enough initial signal change for protein-protein binding study requiring a thrombin concentration of 20 times of the ribozyme/aptamer complex, compared to the 1:1 molar ratio of α-thrombin and aptamer used in this work. With excess bait protein in a competitive assay, a considerable amount of prey protein would be necessary to significantly affect the signal of the aptamer, which may easily lead to false negatives. Using assays, described herein, directly based on aptamers preserve aptamers' affinity to the proteins and monitor protein-protein interactions with high sensitivity.

Identification of Biomarkers and Quantitation of Markers

In a preferred embodiment, a biological sample is obtained from a patient with disease or disorder. Biological samples comprising biomarkers from other patients and control subjects (i.e. normal healthy individuals of similar age, sex, physical condition) are used as comparisons. Biological samples are extracted as discussed above. Preferably, the sample is prepared prior to detection of biomarkers. Typically, preparation involves fractionation of the sample and collection of fractions determined to contain the biomarkers. Methods of pre-fractionation include, for example, size exclusion chromatography ion exchange chromatography, heparin chromatography, affinity chromatography, sequential extraction, gel electrophoresis and liquid chromatography. The analytes also may be modified prior to detection. These methods are useful to simplify the sample for further analysis. For example, it can be useful to remove high abundance proteins, such as albumin, from blood before analysis.

In one embodiment, a sample can be pre-fractionated according to size of proteins in a sample using size exclusion chromatography. For a biological sample wherein the amount of sample available is small, preferably a size selection spin column is used. In general, the first fraction that is eluted from the column (“fraction 1”) has the highest percentage of high molecular weight proteins; fraction 2 has a lower percentage of high molecular weight proteins; fraction 3 has even a lower percentage of high molecular weight proteins; fraction 4 has the lowest amount of large proteins; and so on. Each fraction can then be analyzed by immunoassays, gas phase ion spectrometry, and the like, for the detection of markers.

In another embodiment, a sample can be pre-fractionated by anion exchange chromatography. Anion exchange chromatography allows pre-fractionation of the proteins in a sample roughly according to their charge characteristics. For example, a Q anion-exchange resin can be used (e.g., Q HyperD F, Biosepra), and a sample can be sequentially eluted with eluants having different pH's. Anion exchange chromatography allows separation of biomarkers in a sample that are more negatively charged from other types of biomarkers. Proteins that are eluted with an eluant having a high pH is likely to be weakly negatively charged, and a fraction that is eluted with an eluant having a low pH is likely to be strongly negatively charged. Thus, in addition to reducing complexity of a sample, anion exchange chromatography separates proteins according to their binding characteristics.

In yet another embodiment, a sample can be pre-fractionated by heparin chromatography. Heparin chromatography allows pre-fractionation of the markers in a sample also on the basis of affinity interaction with heparin and charge characteristics. Heparin, a sulfated mucopolysaccharide, will bind markers with positively charged moieties and a sample can be sequentially eluted with eluants having different pH's or salt concentrations. Markers eluted with an eluant having a low pH are more likely to be weakly positively charged. Markers eluted with an eluant having a high pH are more likely to be strongly positively charged. Thus, heparin chromatography also reduces the complexity of a sample and separates markers according to their binding characteristics.

In yet another embodiment, a sample can be pre-fractionated by isolating proteins that have a specific characteristic, e.g. are glycosylated. For example, a CSF sample can be fractionated by passing the sample over a lectin chromatography column (which has a high affinity for sugars). Glycosylated proteins will bind to the lectin column and non-glycosylated proteins will pass through the flow through. Glycosylated proteins are then eluted from the lectin column with an eluant containing a sugar, e.g., N-acetyl-glucosamine and are available for further analysis.

Thus there are many ways to reduce the complexity of a sample based on the binding properties of the proteins in the sample, or the characteristics of the proteins in the sample.

In yet another embodiment, a sample can be fractionated using a sequential extraction protocol. In sequential extraction, a sample is exposed to a series of adsorbents to extract different types of biomarkers from a sample. For example, a sample is applied to a first adsorbent to extract certain proteins, and an eluant containing non-adsorbent proteins (i.e., proteins that did not bind to the first adsorbent) is collected. Then, the fraction is exposed to a second adsorbent. This further extracts various proteins from the fraction. This second fraction is then exposed to a third adsorbent, and so on.

Any suitable materials and methods can be used to perform sequential extraction of a sample. For example, a series of spin columns comprising different adsorbents can be used. In another example, a multi-well comprising different adsorbents at its bottom can be used. In another example, sequential extraction can be performed on a probe adapted for use in a gas phase ion spectrometer, wherein the probe surface comprises adsorbents for binding biomarkers. In this embodiment, the sample is applied to a first adsorbent on the probe, which is subsequently washed with an eluant. Markers that do not bind to the first adsorbent are removed with an eluant. The markers that are in the fraction can be applied to a second adsorbent on the probe, and so forth. The advantage of performing sequential extraction on a gas phase ion spectrometer probe is that markers that bind to various adsorbents at every stage of the sequential extraction protocol can be analyzed directly using a gas phase ion spectrometer.

In yet another embodiment, biomarkers in a sample can be separated by high-resolution electrophoresis, e.g., one or two-dimensional gel electrophoresis. A fraction containing a marker can be isolated and further analyzed by gas phase ion spectrometry.

Preferably, two-dimensional gel electrophoresis is used to generate two-dimensional array of spots of biomarkers, including one or more markers. See, e.g., Jungblut and Thiede, Mass Spectr. Rev. 16:145-162 (1997).

The two-dimensional gel electrophoresis can be performed using methods known in the art. See, e.g., Deutscher ed., Methods In Enzymology vol. 182. Typically, biomarkers in a sample are separated by, e.g., isoelectric focusing, during which biomarkers in a sample are separated in a pH gradient until they reach a spot where their net charge is zero (i.e., isoelectric point). This first separation step results in one-dimensional array of biomarkers. The biomarkers in one dimensional array is further separated using a technique generally distinct from that used in the first separation step. For example, in the second dimension, biomarkers separated by isoelectric focusing are further separated using a polyacrylamide gel, such as polyacrylamide gel electrophoresis in the presence of sodium dodecyl sulfate (SDS-PAGE). SDS-PAGE gel allows further separation based on molecular mass of biomarkers. Typically, two-dimensional gel electrophoresis can separate chemically different biomarkers in the molecular mass range from 1000-200,000 Da within complex mixtures.

Biomarkers in the two-dimensional array can be detected using any suitable methods known in the art. For example, biomarkers in a gel can be labeled or stained (e.g., Coomassie Blue or silver staining). If gel electrophoresis generates spots that correspond to the molecular weight of one or more markers of the invention, the spot can be further analyzed by densitometric analysis or gas phase ion spectrometry. For example, spots can be excised from the gel and analyzed by gas phase ion spectrometry. Alternatively, the gel containing biomarkers can be transferred to an inert membrane by applying an electric field. Then a spot on the membrane that approximately corresponds to the molecular weight of a marker can be analyzed by gas phase ion spectrometry. In gas phase ion spectrometry, the spots can be analyzed using any suitable techniques, such as MALDI or SELDI.

Prior to gas phase ion spectrometry analysis, it may be desirable to cleave biomarkers in the spot into smaller fragments using cleaving reagents, such as proteases (e.g., trypsin). The digestion of biomarkers into small fragments provides a mass fingerprint of the biomarkers in the spot, which can be used to determine the identity of markers if desired.

In yet another embodiment, high performance liquid chromatography (HPLC) can be used to separate a mixture of biomarkers in a sample based on their different physical properties, such as polarity, charge and size. HPLC instruments typically consist of a reservoir of mobile phase, a pump, an injector, a separation column, and a detector. Biomarkers in a sample are separated by injecting an aliquot of the sample onto the column. Different biomarkers in the mixture pass through the column at different rates due to differences in their partitioning behavior between the mobile liquid phase and the stationary phase. A fraction that corresponds to the molecular weight and/or physical properties of one or more markers can be collected. The fraction can then be analyzed by gas phase ion spectrometry to detect markers.

Optionally, a marker can be modified before analysis to improve its resolution or to determine its identity. For example, the markers may be subject to proteolytic digestion before analysis. Any protease can be used. Proteases, such as trypsin, that are likely to cleave the markers into a discrete number of fragments are particularly useful. The fragments that result from digestion function as a fingerprint for the markers, thereby enabling their detection indirectly. This is particularly useful where there are markers with similar molecular masses that might be confused for the marker in question. Also, proteolytic fragmentation is useful for high molecular weight markers because smaller markers are more easily resolved by mass spectrometry. In another example, biomarkers can be modified to improve detection resolution. For instance, neuraminidase can be used to remove terminal sialic acid residues from glycoproteins to improve binding to an anionic adsorbent and to improve detection resolution. In another example, the markers can be modified by the attachment of a tag of particular molecular weight that specifically bind to molecular markers, further distinguishing them. Optionally, after detecting such modified markers, the identity of the markers can be further determined by matching the physical and chemical characteristics of the modified markers in a protein database (e.g., SwissProt).

After preparation, biomarkers in a sample are typically captured on a substrate for detection. Traditional substrates include antibody-coated 96-well plates or nitrocellulose membranes that are subsequently probed for the presence of proteins. Preferably, the biomarkers are identified using immunoassays as described above. However, preferred methods also include the use of biochips. Preferably the biochips are protein biochips for capture and detection of proteins. Many protein biochips are described in the art. These include, for example, protein biochips produced by Packard BioScience Company (Meriden Conn.), Zyomyx (Hayward, Calif.) and Phylos (Lexington, Mass.). In general, protein biochips comprise a substrate having a surface. A capture reagent or adsorbent is attached to the surface of the substrate. Frequently, the surface comprises a plurality of addressable locations, each of which location has the capture reagent bound there. The capture reagent can be a biological molecule, such as a polypeptide or a nucleic acid, which captures other biomarkers in a specific manner. Alternatively, the capture reagent can be a chromatographic material, such as an anion exchange material or a hydrophilic material. Examples of such protein biochips are described in the following patents or patent applications: U.S. Pat. No. 6,225,047 (Hutchens and Yip, “Use of retentate chromatography to generate difference maps,” May 1, 2001), International publication WO 99/51773 (Kuimelis and Wagner, “Addressable protein arrays,” October 14, 1999), International publication WO 00/04389 (Wagner et al., “Arrays of protein-capture agents and methods of use thereof,” Jul. 27, 2000), International publication WO 00/56934 (Englert et al., “Continuous porous matrix arrays,” Sep. 28, 2000).

In general, a sample containing the biomarkers is placed on the active surface of a biochip for a sufficient time to allow binding. Then, unbound molecules are washed from the surface using a suitable eluant. In general, the more stringent the eluant, the more tightly the proteins must be bound to be retained after the wash. The retained protein biomarkers now can be detected by appropriate means.

Analytes captured on the surface of a protein biochip can be detected by any method known in the art. This includes, for example, mass spectrometry, fluorescence, surface plasmon resonance, ellipsometry and atomic force microscopy. Mass spectrometry, and particularly SELDI mass spectrometry, is a particularly useful method for detection of the biomarkers of this invention.

Preferably, a laser desorption time-of-flight mass spectrometer is used in embodiments of the invention. In laser desorption mass spectrometry, a substrate or a probe comprising markers is introduced into an inlet system. The markers are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of markers of specific mass to charge ratio.

Matrix-assisted laser desorption/ionization mass spectrometry, or MALDI-MS, is a method of mass spectrometry that involves the use of an energy absorbing molecule, frequently called a matrix, for desorbing proteins intact from a probe surface. MALDI is described, for example, in U.S. Pat. No. 5,118,937 (Hillenkamp et al.) and U.S. Pat. No. 5,045,694 (Beavis and Chait). In MALDI-MS the sample is typically mixed with a matrix material and placed on the surface of an inert probe. Exemplary energy absorbing molecules include cinnamic acid derivatives, sinapinic acid (“SPA”), cyano hydroxy cinnamic acid (“CHCA”) and dihydroxybenzoic acid. Other suitable energy absorbing molecules are known to those skilled in this art. The matrix dries, forming crystals that encapsulate the analyte molecules. Then the analyte molecules are detected by laser desorption/ionization mass spectrometry. MALDI-MS is useful for detecting the biomarkers of this invention if the complexity of a sample has been substantially reduced using the preparation methods described above.

Surface-enhanced laser desorption/ionization mass spectrometry, or SELDI-MS represents an improvement over MALDI for the fractionation and detection of biomolecules, such as proteins, in complex mixtures. SELDI is a method of mass spectrometry in which biomolecules, such as proteins, are captured on the surface of a protein biochip using capture reagents that are bound there. Typically, non-bound molecules are washed from the probe surface before interrogation. SELDI is described, for example, in: U.S. Pat. No. 5,719,060 (“Method and Apparatus for Desorption and Ionization of Analytes,” Hutchens and Yip, Feb. 17, 1998,) U.S. Pat. No. 6,225,047 (“Use of Retentate Chromatography to Generate Difference Maps,” Hutchens and Yip, May 1, 2001) and Weinberger et al., “Time-of-flight mass spectrometry,” in Encyclopedia of Analytical Chemistry, R. A. Meyers, ed., pp 11915-11918 John Wiley & Sons Chichesher, 2000.

Markers on the substrate surface can be desorbed and ionized using gas phase ion spectrometry. Any suitable gas phase ion spectrometers can be used as long as it allows markers on the substrate to be resolved. Preferably, gas phase ion spectrometers allow quantitation of markers.

In one embodiment, a gas phase ion spectrometer is a mass spectrometer. In a typical mass spectrometer, a substrate or a probe comprising markers on its surface is introduced into an inlet system of the mass spectrometer. The markers are then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of the presence of markers or other substances will typically involve detection of signal intensity. This, in turn, can reflect the quantity and character of markers bound to the substrate. Any of the components of a mass spectrometer (e.g., a desorption source, a mass analyzer, a detector, etc.) can be combined with other suitable components described herein or others known in the art in embodiments of the invention.

In another embodiment, an immunoassay can be used to detect and analyze markers in a sample. This method comprises: (a) providing an aptamer that specifically binds to a marker; (b) contacting a sample with the aptamer; and (c) detecting the presence of a complex of the aptamer bound to the marker in the sample.

To prepare an aptamer that specifically binds to a marker, purified markers or their nucleic acid sequences can be used. Nucleic acid and amino acid sequences for markers can be obtained by fuirther characterization of these markers. For example, each marker can be peptide mapped with a number of enzymes (e.g., trypsin, V8 protease, etc.). The molecular weights of digestion fragments from each marker can be used to search the databases, such as SwissProt database, for sequences that will match the molecular weights of digestion fragments generated by various enzymes. Using this method, the nucleic acid and amino acid sequences of other markers can be identified if these markers are known proteins in the databases.

Alternatively, the proteins can be sequenced using protein ladder sequencing. Protein ladders can be generated by, for example, fragmenting the molecules and subjecting fragments to enzymatic digestion or other methods that sequentially remove a single amino acid from the end of the fragment. Methods of preparing protein ladders are described, for example, in International Publication WO 93/24834 (Chait et al.) and U.S. Pat. No. 5,792,664 (Chait et al.). The ladder is then analyzed by mass spectrometry. The difference in the masses of the ladder fragments identify the amino acid removed from the end of the molecule.

If the markers are not known proteins in the databases, nucleic acid and amino acid sequences can be determined with knowledge of even a portion of the amino acid sequence of the marker. For example, degenerate probes can be made based on the N-terminal amino acid sequence of the marker. These probes can then be used to screen a genomic or cDNA library created from a sample from which a marker was initially detected. The positive clones can be identified, amplified, and their recombinant DNA sequences can be subdloned using techniques which are well known. See, e.g., Current Protocols for Molecular Biology (Ausubel et al., Green Publishing Assoc. and Wiley-Interscience 1989) and Molecular Cloning: A Laboratory Manual, 3rd Ed. (Sambrook et al., Cold Spring Harbor Laboratory, NY 2001).

Using the purified markers or their nucleic acid sequences, aptamers that specifically bind to a marker can be prepared using any suitable methods known in the art. A typical aptamer preparation has been discussed above.

After the aptamer is provided, a marker can be detected and/or quantified using any suitable binding assays known in the art (see, e.g., the Examples which follow). These aptamers can also be correlated with antibody binding if desired. Useful assays include, for example, an enzyme immune assay (EIA) such as enzyme-linked immunosorbent assay (ELISA), a radioimmune assay (RIA), a Western blot assay, or a slot blot assay. These methods are also described in, e.g., Methods in Cell Biology: Antibodies in Cell Biology, volume 37 (Asai, ed. 1993); Basic and Clinical Immunology (Stites & Terr, eds., 7th ed. 1991); and Harlow & Lane, supra.

Generally, a sample obtained from a subject can be contacted with the aptamer(s) that specifically bind(s) the marker. Optionally, the aptamer can be fixed to a solid support to facilitate washing and subsequent isolation of the complex, prior to contacting the aptamer with a sample. Examples of solid supports include glass or plastic in the form of, e.g., a microtiter plate, a stick, a bead, or a microbead. Aptamers can also be attached to a probe substrate or chip arrays described above. The sample is preferably a biological fluid sample taken from a subject. Examples of biological fluid samples include cerebrospinal fluid, blood, serum, plasma, neuronal cells, tissues, urine, tears, saliva etc. The sample can be diluted with a suitable eluant before contacting the sample to the aptamer.

After incubating the sample with aptamers, the mixture is washed and the aptamer-marker complex formed can be detected. This can be accomplished by incubating the washed mixture with a detection reagent. This detection reagent may be, e.g., an antibody which is labeled with a detectable label or directly labeled as described in the prey-bait assay described in detail in the Examples which follow. Exemplary detectable labels include magnetic beads (e.g., DYNABEADS™), fluorescent dyes, radiolabels, enzymes (e.g., horse radish peroxide, alkaline phosphatase and others commonly used in an ELISA), and calorimetric labels such as colloidal gold or colored glass or plastic beads.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

The fluorescence anisotropy assays described herein can be used to determine presence or absence of a marker in a sample as well as the quantity of a marker in a sample. First, a test amount of a marker in a sample can be detected using the methods described in the Examples which follow. For example, in a competitive assay, the interaction of aptamer and its target protein is are, if desired, compared to a known interaction. The addition of the prey protein may shift the equilibrium of the aptamer/bait protein binding reaction and cause a signal change. Using the aptamer/α-thrombin interaction, the assay can be conducted as follows. Based on the known aptamer/α-thrombin interaction and equilibrium conditions, the K_{d} of αa-thrombin/prey protein binding reaction is calculated using a single signal change that occurred when the prey protein was added to the aptamer/α-thrombin complex solution.

Assume C_{A} molar of T-15Ap aptamer and C_{T} molar of α-thrombin are mixed together. When C_{P} molar of prey protein is added to the mixture, it displaces T-15Ap and result in a decreased anisotropy value of r_{new}. r_{new} can be represented using the following equation:

r_{A}·x+r_{AT}·(1−x)=_{new}

where r_{A} and r_{AT} are anisotropies of the two fluorescent species in the solution, T-15Ap and T-15Ap/α-thrombin complex respectively, and x is fraction of the unbound T-15Ap aptamer. Since r_{A} and r_{AT} are known properties of the aptamer/α-thrombin system and r_{new} is the measured new anisotropy, it is easy to find out that:

$$x = \frac{{r}_{new} - {r}_{AT}}{{r}_{A} - {r}_{AT}}$$

Then the concentrations of unbound and bound T-15Ap are:

[T15Ap]=C_{A}·x [T-15Ap/α-thrombin]=C_{A}·(1−x)

Because the dissociation constant of aptamer/α-thrombin reaction (K_{d/AT}) is already known, then:

$$[ α - thrombin ] = \frac{{K}_{d / AT} \cdot [ T - 15 Ap / α - thrombin ]}{[ T - 15 Ap ]}$$

Since C_{T}=[α-thrombin]+[T-15Ap/α-thrombin]+[prey/α-thrombin], [prey/α-thrombin]=C_{T}−[α-thrombin]−[T-15Ap/α-thrombin]

Similarly, C_{P}=[prey/α-thrombin]+[preyprotein], so [prey protein]=C_{P}−[prey/α-thrombin]

Finally, the dissociation constant of α-thrombin/prey protein binding reaction (K_{d/TP}) is given by the following equation:

$${K}_{d / TP} = \frac{[ prey protein ] \cdot [ α - thrombin ]}{[ prey / α - thrombin ]}$$

Using a simple computer program, it is possible to routinely calculate protein-protein binding affinity using data obtained from the aptamer-based competitive assay for protein-protein interactions.

An isotropy Measurements:Anisotropy measurements were based on the following equation:

$$Anisotropy r = \frac{{I}_{VV} - G \cdot {I}_{VH}}{{I}_{VV} + 2 G \cdot {I}_{VH}}$$

where the subscripts V and H refer to the orientation (vertical or horizontal) of the polarizers for the intensity measurements, with the first subscript indicating the position of the excitation polarizer and the second for the emission polarizer. G is the G-factor of the spectrofluorometer, which is calculated as G=I_{HV/I}_{HH}. The G-factor represents the ratio of the sensitivities of the detection system for vertically and horizontally polarized light, and is dependent on the emission wavelength. For a certain dye, the G-factor would be measured and used throughout the experiments that used the same dye. Then the spectrofluorometer would keep the excitation polarizer vertical and rotate the emission polarizer from vertical to horizontal position to measure the intensities for anisotropy calculation. For TAMRA, all intensities were measured at an emission wavelength of 580 nm with an excitation wavelength of 555 nm. Time-based anisotropy measurements were carried out by continuously monitoring anisotropy every couple of minutes. With an integration time of 1.5 seconds, each anisotropy measurement takes about 6.1 seconds. Thus, if a marker is present in the sample, the aptamer-marker association can be detected and calculated. A standard can be, e.g., a known compound or another protein known to be present in a sample. As noted above, the test amount of marker need not be measured in absolute units, as long as the unit of measurement can be compared to a control.

Data generated by desorption and detection of markers can be analyzed using any suitable means. In one embodiment, data is analyzed with the use of a programmable digital computer. The computer program generally contains a readable medium that stores codes. Certain code can be devoted to memory that includes the location of each feature on a probe, the identity of the adsorbent at that feature and the elution conditions used to wash the adsorbent. The computer also contains code that receives as input, data on the strength of the signal at various molecular masses received from a particular addressable location on the probe. This data can indicate the number of markers detected, including the strength of the signal generated by each marker.

Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their fall scope of equivalents.

All publications and patent documents cited in this application are incorporated by reference in pertinent part for all purposes to the same extent as if each individual publication or patent document were so individually denoted. By their citation of various references in this document, Applicants do not admit any particular reference is “prior art” to their invention.

Without further elaboration, it is believed that one skilled in the art can, using the preceding description, utilize the present invention to its fullest extent. The invention will be further clarified by a consideration of the following examples, which are intended to be purely exemplary of the present invention are thus to be construed as merely illustrative examples and not limitations of the scope of the present invention in any way.

### EXAMPLES

### Materials and Methods

Materials

Dye-labeled aptamers were obtained from Integrated DNA Technologies, Inc. (Coralville, Iowa). The sequences of the 15mer and 27mer thrombin-aptamer are 5′-GGT TGG TGT GGT TGG-3′ (SEQ ID NO 1), and 5′-ACC CGT GGT AGG GTA GGA TGG GGT GGT-3′ (SEQ ID NO 2) respectively. For FRET-based assays, both aptamers were dual-labeled with 6-FAM at the 5′ end and Dabcyl at the 3′ end. For fluorescence anisotropy assays, both aptamer sequences were labeled with only TAMRA at the 3′ end. A control 15mer aptamer was labeled with only 6-FAM at the 3′ end. All aptamers were purified with HPLC.

Human oa-thrombin (M.W. ˜36.7 kDa), human antitlrombin mI (.W. ˜58 kDa) and a monoclonal antibody anti-human thrombin (M.W. ˜150 kDa) were obtained from Haematologic Technologies Inc. (Essex Junction, Vt.). Bovine serum albumin (BSA) (M.W. ˜67 kDa) and a sulfated hirudin fragment 54-65, Gly-Asp-Phe-Glu-Glu-Ile-Pro-Glu-Glu-Tyr(SO₃H)-Leu-Gln (SEQ ID NO: 5)(M.W. ˜1.5 kDa), were from Sigma-Aldrich, Inc. (St. Louis, Mo.). All tests were performed in a 20 mM Tris-HCl buffer with a pH of 7.6 that contained 50 mM NaCl and 5% (V/V) glycerol. All reagents for the buffer were obtained from Fisher Scientific Company L.L.C. (Pittsburgh, Pa.).

Fluorescence FRET and Anisotropy Measurements.

Fluorescence measurements were performed on a Fluorolog-3 spectrofluorometer (Jobin Yvon Inc., Edison, N.J.). For FRET-based assays, the fluorescence of 6-FAM was monitored with an excitation wavelength of 488 mn and an emission wavelength of 515 nm. For anisotropy-based experiments, the fluorescence of TAMRA was monitored with 555 nm as the excitation and 580 mn as the emission wavelength. Slit widths were varied to yield the best signals. All measurements were carried out in a 100 μL cuvette. In the aptamer/thrombin binding experiments, a very small volume of α-thrombin at a high concentration was added to an aptamer solution in the cuvette to make a molar ratio of aptamer and thrombin 1:1, and the fluorescence signals were recorded before and after the addition. For protein-protein binding reaction, an aptamer/thrombin mixture at 1:1 molar ratio was placed in the cuvette, and small volumes of the second protein solution at high concentrations were added to the mixture to make the desired prey protein concentrations. All dilution effects caused by the addition of samples to the original solutions were corrected during data analysis. Anisotropy measurements were also done with Fluorolog-3 spectrofluorometer. Gel electrophoresis was carried out based on standard procedures.

Kinetic Studies.

Experiments were conducted in a 100 μL cuvette in the spectrofluorometer. While the detection system was running, the reaction samples were quickly mixed together. Data were recorded from the point of mixing to when the signal reached the plateau and stabilized. The reaction was regarded completed when the signal was at the plateau. Detections were either done using steady state anisotropy measurements for AT3 and AHT study with T-15Ap, or steady state fluorescence measurements for HirF study with FQ-15Ap. Study with AT3 was conducted at room temperature while AHT and HirF experiments were done at 5° C. The temperatures of reactions were maintained using a RTE-111 water bath/circulator (Neslab Instruments, Inc., Newington, N.H.).

Calculation of Kd of Protein-Protein Interaction in the Competitive Assay.

In a competitive assay, such as described in this work, the interaction of aptamer and its target protein is a known system. The addition of the prey protein may shift the equilibrium of the aptamer/bait protein binding reaction and cause a signal change. Based on the known aptamer/α-thrombin interaction and equilibrium conditions, it was possible to calculate the K_{d} of α-thrombin/prey protein binding reaction using a single signal change that occurred when the prey protein was added to the aptamer/α-thrombin complex solution.

Assume C_{A} molar of T-15Ap aptamer and C_{T} molar of α-thrombin are mixed together. When Cp molar of prey protein is added to the mixture, it will displace T-15Ap and result in a decreased anisotropy value of r_{new}. r_{new} can be represented using the following equation:

r_{A}·x+r_{AT}·(1−x)=r_{new}

where r_{A} and r_{AT} are anisotropies of the two fluorescent species in the solution, T-15Ap and T-15Ap/α-thrombin complex respectively, and x is fraction of the unbound T-15Ap aptamer. Since r_{A} and r_{AT} are known properties of the aptamer/α-thrombin system and r_{new} is the measured new anisotropy, it is easy to find out that:

$$x = \frac{{r}_{new} - {r}_{AT}}{{r}_{A} - {r}_{AT}}$$

Then the concentrations of unbound and bound T-15Ap are:

[T15Ap]=C_{A}·x [T-15Ap/α-thrombin]=C_{A}·(1−x)

Because the dissociation constant of aptamer/α-thrombin reaction (K_{d/AT}) is already known, then:

$$[ α - thrombin ] = \frac{{K}_{d / AT} \cdot [ T - 15 Ap / α - thrombin ]}{[ T - 15 Ap ]}$$

Since C_{T}=[α-thrombin]+[T-15Ap/α-thrombin]+[prey/α-thrombin], [prey/α-thrombin]=C_{T}−[α-thrombin]−[T-15Ap/α-thrombin]

Similarly, C_{P}=[prey/α-thrombin]+[prey protein], so [prey protein]=C_{P}−[prey/α-thrombin]

Finally, the dissociation constant of α-thrombin/prey protein binding reaction (K_{d/TP}) is given by the following equation:

$${K}_{d / TP} = \frac{[ prey protein ] \cdot [ α - thrombin ]}{[ prey / α - thrombin ]}$$

Using a simple computer program, it is possible to routinely calculate protein-protein binding affinity using data obtained from the aptamer-based competitive assay for protein-protein interactions.

Ainisotropy Measurements.

Anisotropy measurements were based on the following equation:

$$Anisotropy r = \frac{{I}_{VV} - G \cdot {I}_{VH}}{{I}_{VV} + 2 G \cdot {I}_{VH}}$$

where the subscripts V and H refer to the orientation (vertical or horizontal) of the polarizers for the intensity measurements, with the first subscript indicating the position of the excitation polarizer and the second for the emission polarizer. G is the G-factor of the spectrofluorometer, which is calculated as G=I_{HV}/I_{HH}. The G-factor represents the ratio of the sensitivities of the detection system for vertically and horizontally polarized light, and is dependent on the emission wavelength. For a certain dye, the G-factor would be measured and used throughout the experiments that used the same dye. Then the spectrofluorometer would keep the excitation polarizer vertical and rotate the emission polarizer from vertical to horizontal position to measure the intensities for anisotropy calculation. For TAMRA, all intensities were measured at an emission wavelength of 580 nm with an excitation wavelength of 555 nm. Time-based anisotropy measurements were carried out by continuously monitoring anisotropy every couple of minutes. With an integration time of 1.5 seconds, each anisotropy measurement takes about 6.1 seconds.

Gel Electrophoresis.

Gel electrophoresis was performed on a Mini-Protean® 3 precast gel system (Bio-Rad Laboratories, Inc., Hercules, Calif.). Samples loaded on a 7.5% resolving Tris-HCl native gel (Bio-Rad Laboratories, Inc., Hercules, Calif.) were run at 150 V for 150 minutes. The gel was then taken out, rinsed with ultra-pure water and stained with Coomassie blue stain reagent (Fisher Scientific Company L.L.C., Pittsburgh, Pa.) for 1 hour. A digital camera was used to image the stained gel.

### Example 1

FRET-Based Signaling Aptamer for Protein Binding.

Human α-thrombin (α-thrombin) and its aptamers were used to demonstrate the capability of aptamers to probe protein-protein interactions. α-thrombin has two positive-charged sites termed Exosite I and II on the opposite sides of the protein. Exosite I was found to bind to fibrinogen and hirudin while Exosite II binds to a serine protease inhibitor antithrombin III (AT3). Two different aptamers have been identified that have high affinity and selectivity for α-thrombin. The first one is a 15mer single-stranded DNA aptamer which binds to the fibrinogen-binding site of oa-thrombin, namely Exosite I. The other DNA aptamer, with a 27mer backbone length, was determined to bind to the Exosite II of α-thrombin. Both aptamers were found to adopt a G-quartet structure when bound to α-thrombin. A 15mer Exosite I binding aptamer (15Ap, Table 1) and a 27mer Exosite I binding aptamer (27Ap, Table 1) with similar thrombin-binding affinity were chosen to study the interactions of α-thrombin with other proteins.

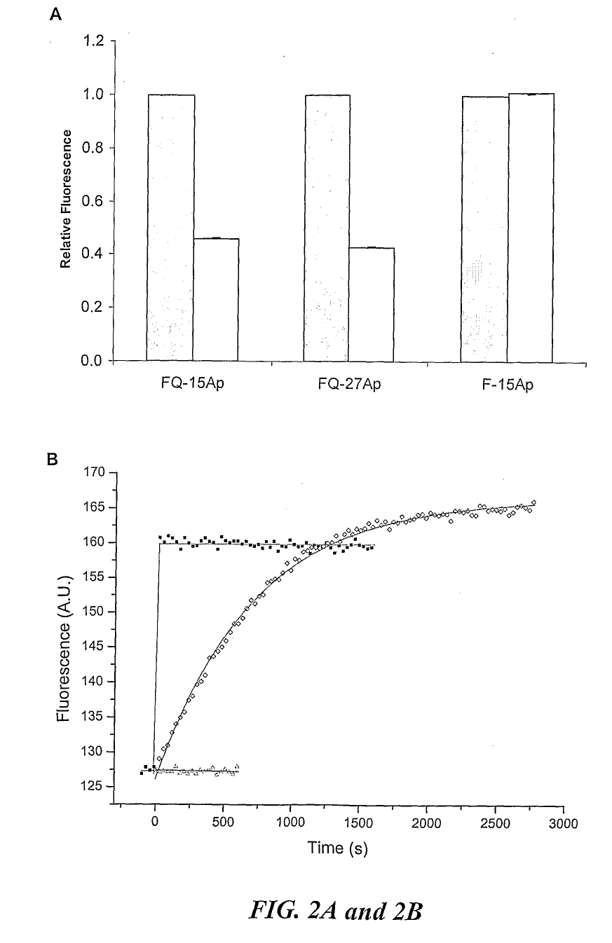

We previously reported a molecular beacon aptamer for α-thrombin detection based on the 15Ap (Li, J. W. J., Fang, X. H. & Tan, W. H. (2002) Biochem. Biophys. Res. Commun. 292, 31-40; Fang, X. H., Sen, A., Vicens, M. & Tan, W. H. (2003) ChemBioChem, 4, 829-834). Here a modified aptamer (FQ-15Ap, Table 1) has been used that incorporates a 6-carboxyfluorescein (6-FAM) at the 5′ end of the DNA as the donor and a Dabcyl at the 3′ end as the quencher. The quenching of 6-FAM emission is caused by energy transfer between 6-FAM and Dabcyl in the protein-binding induced G-quartet structure where the two labels are in close proximity. When excess α-thrombin was added to an FQ-15Ap solution at room temperature, the fluorescence of 6-FAM dropped about 55 percent (FIG. 2A). High metal ion concentrations, especially the presence of K⁺, can promote the formation of G-quartet, which results in a much lower fluorescence signal change upon aptamer/α-thrombin binding. However, using a buffer without any metal ions was found to inhibit protein-protein interactions. By keeping a 50 mM NaCl concentration in the buffer, the protein activities were sustained and relatively high fluorescence quenching induced by protein binding to the aptamer was found. When α-thrombin was added to a control 15mer aptamer that was labeled only with 6-FAM, no significant fluorescence change was observed (FIG. 2A), indicating that the fluorescence decrease in the FQ-15Ap-thrombin binding experiment was due to the binding-induced conformational change of the aptamer rather than a direct quenching of the dye 6-FAM by α-thrombin. Quenching was not observed (i) under conditions where thrombin would not bind the aptamer, and (ii) with a scrambled aptamer to which thrombin does not bind.

### Example 2

Dual-Labeled Aptamer for Thrombin-Protein Binding Study.

The 1:1 molar ratio FQ-15Ap/α-thrombin solution (bait solution) was used to identify interactions of α-thrombin with other proteins. When a second protein (prey protein) binds to the same site of α-thrombin as the FQ-15Ap, the aptamer is thought to be displaced and the freed aptamer shifts back to a more relaxed conformation, resulting in restored 6-FAM fluorescence. A sulfated fragment of hirudin that contained the C-terminal 13-residue (HirF) instead of hirudin was used for binding α-thrombin. The addition of HirF to the FQ-15Ap bait solution caused a sharp fluorescence increase (FIG. 2B), since both HirF and FQ-15Ap bound to the same site of α-thrombin. Control experiments showed that there was no fluorescence change when HirF was added to a FQ-15Ap in the absence of thrombin, indicating that there was no direct interaction between the aptamer and HirF. The time course results showed that this competitive binding reaction was fast as the aptamer departed within seconds after HirF was added to the aptamer-thrombin complex solution.

Several other proteins were also investigated for interactions with α-thrombin using the FQ-15Ap bait solution. The addition of an antibody, anti-human thrombin (AHT), caused no significant change in the fluorescence of 6-FAM (FIG. 2B). While this result indicates that AHT does not compete with the aptamer for the Exosite I of α-thrombin, it cannot exclude the possibility that AHT still binds to α-thrombin but at a different site of α-thrombin. Antithrombin III (AT3) was also tested in the bait solution. A slow-signal increasing trend was observed for AT3 (FIG. 2B). Addition of excess AT3 further increased the 6-FAM fluorescence, but the fluorescence intensity never exceeded that of the FQ-15Ap solution in the absence of α-thrombin. This result could be explained in that the binding of AT3 to α-thrombin may have caused a conformational change in α-thrombin that rendered the binding with the aptamer at Exosite I unstable.

Bovine serum albumin (BSA) was used as a control protein for interaction with α-thrombin. No fluorescence change was observed for BSA. Another set of control experiments were conducted by adding the prey proteins to be tested to an FQ-15Ap buffer solution without α-thrombin. None of the proteins affected fluorescence of the aptamer, meaning they did not interact with either the aptamer or the fluorophore.

It is also possible to quantify the amount of prey protein that is interacting with thrombin using a different level of signal change. It was found that at higher thrombin to aptamer ratio such as 2: 1, it took more prey protein to cause similar quantity of signal change, thus diminishing the sensitivity of this assay. For that reason, 1:1 ratio of thrombin and aptamer was used in all the experiments.

### Example 3

FRET-Based 27mer Aptamerfor Thrombin-Protein Binding.

The sequence of the Exosite II-binding 27mer aptamer was adopted from a previous report. The aptamer was labeled with 6-FAM and Dabcyl similar to FQ-15Ap. With the addition of α-thrombin, FQ-27Ap also displayed decreased 6-FAM fluorescence because 6-FAM and Dabcyl at the two ends of the aptamer were brought closer in the quadruplex structure. The relative fluorescence decrease was found to be a little larger than that in the FQ-15Ap experiments (FIG. 2A). Compared to the noise level, the absolute fluorescence difference between the bound and the unbound FQ-27Ap provided adequate sensitivity for the thrombin-protein interaction study.

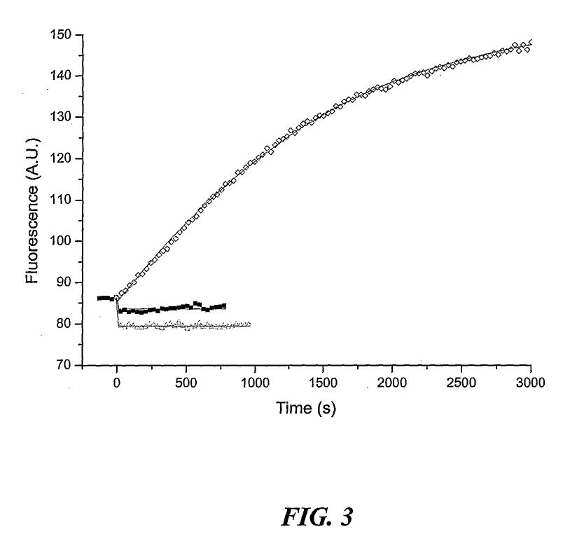

Different proteins were investigated in a FQ-27Ap/α-thrombin bait solution in a similar way as in the FQ-15Ap based assay. The results for HirF and AHT showed slightly decreased signals (FIG. 3), indicating no displacement of FQ-27Ap took place. The fluorescence reduction could be caused by interactions of thrombin with those two molecules. In contrast, antithrombin III still displayed a gradual increase in 6-FAM fluorescence. The results indicate that the interaction between AT3 and α-thrombin is a relatively slow process.

### Example 4

Fluorescence Anisotropy (FA) Based Aptamer Probes for Protein Interactions.

To address some of the unresolved problems in FRET experiments such as how AT3 really binds to α-thrombin and what happens between AHT and α-thrombin, a complementary strategy was developed based on fluorescence anisotropy. Fluorescence anisotropy is widely used for studying the interactions of biomolecules due to its capability of sensing changes in molecular size or molecular weight. The thrombin aptamers were labeled with only one TAMRA dye at the 3′ end to create anisotropy aptamer probes, the 15mer T-15Ap and the 27mer T-27Ap (Table 1).

TABLE 1

Sequences of the fluorophore-labeled aptamers.

| | |
|---|---|
| Oligo name | Oligo sequence |

| | | |
|---|---|---|
| FQ-15Ap | 5′-(6-FAM)-GGT TGG TGT GGT TGG-(Dabcyl)-3′ | (SEQ ID NO: 1) |
| T-15Ap | 5′-GGT TGG TGT GGT TGG-(TAMRA)-3′ | (SEQ ID NO: 2) |
| FQ-27Ap | 5′-(6-FAM)-ACC CGT GGT AGG GTA GGA TGG GGT GGT- | (SEQ ID NO: 3) |
| | (Dabcyl)-3′ | |
| T-27Ap | 5′-ACC CGT GGT AGG GTA GGA TGG GGT GGT--(TAMRA)- | (SEQ ID NO: 4) |
| | 3′ | |

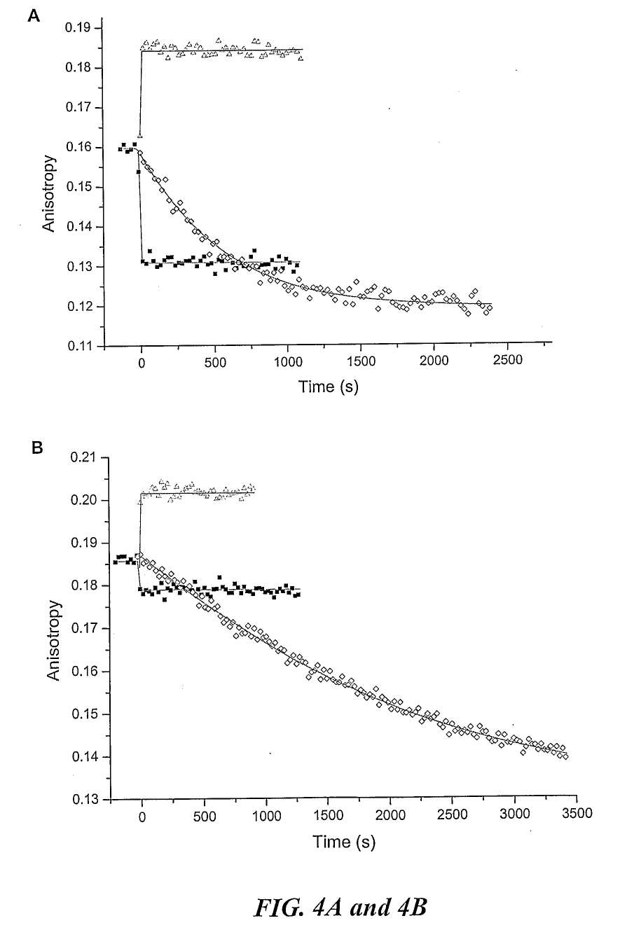

The T-15Ap was first investigated for its ability to probe protein interactions. When T-15Ap/α-thrombin (1:1) solutions were mixed together, the anisotropy of T-15Ap increased more than 30%. This bait solution was then tested with different prey proteins (FIG. 4A). The anisotropy dropped within seconds upon addition of HirF to the bait solution and remained almost constant after that. This result correlates well with the result from the FRET-based experiment. Without wishing to be bound by theory, this may be explained as a quick displacement of the aptamer by HirF at the Exosite I binding site of α-thrombin. The anisotropy decreased as a result of the increased concentration of unbound aptamer which had a much lower molecular weight than that of the aptamer-protein complex. The reaction was rapid, indicating a simple binding between HirF and a-thrombin through non-covalent bonds.

The AT3 curve showed a different decreasing trend with time. It was rather slow and gradual, similar to the FRET-based result. In the FRET assay, it clearly illustrated that the aptamer was displaced. There could be several pathways that the AT3/α-thrombin interaction might have taken. One of them is that all the AT3 molecules would quickly bind to Exosite II of α-thrombin, and a slow conformational change of α-thrombin induced by AT3 binding then caused FQ-15Ap to leave Exosite I. In another pathway, AT3 would slowly attack Exosite II and while this was happening, the aptamer would leave α-thrombin. The FRET-based method could not differentiate between these two mechanisms. On the other hand, using fluorescence anisotropy, if the AT3/α-thrombin interaction underwent the first pathway, the increased molecular weight through the binding of AT3 to α-thrombin/aptamer complex in the first step would introduce an initial anisotropy increase. Then , the anisotropy would slowly decrease from that point on as the T-15Ap slowly became unbound. However, the real time anisotropy detection of the AT3/α-thrombin interaction (FIG. 4A) demonstrated no such initial anisotropy jump. Combined with the result from FQ-27Ap, it can be seen, without wishing to be bound by theory, that the second pathway is more likely to be the mechanism for this protein-protein interaction. The anisotropy approach is shown here to be able to provide insight into the kinetics and mechanisms of the targeted interactions, which will be highly useful in understanding proteins' functions. Site-directed aptamers enable real-time, ultrasensitive studies on protein-protein interaction.

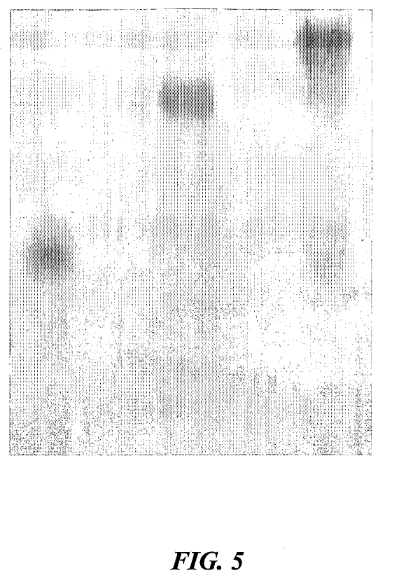

AHT caused an immediate anisotropy increase of T-15Ap when added to the aptamer/α-thrombin bait solution (FIG. 4A). While the lack of a decreased anisotropy correlated with the FRET-based result that showed AHT had no effect on binding between the 15mer aptamer and α-thrombin, the anisotropy increase suggested the presence of a binding between AHT and α-thrombin. Furthermore, this binding happened at a different site than Exosite I, which added extra weight to the aptamer/α-thrombin complex. The binding of AHT and α-thrombin was further confirmed using gel electrophoresis (FIG. 5). One advantage of the anisotropy-based method over the FRET-based method and many other techniques is that it can differentiate interactions at different binding sites.

Bait solutions containing T-27Ap and α-thrombin were also used to probe protein-protein interactions at the Exosite II of α-thrombin (FIG. 4B). HirF caused a slightly lower anisotropy change even though it binds to Exosite I. Considering HirF is a rather small molecule (M.W.=˜1.5 KDa), the small anisotropy decrease was likely caused by HirF displacing T-27Ap. However, this displacement was much smaller compared to that of T-15Ap. AT3 displayed a gradually decreasing anisotropy as it slowly replaced T-27Ap. In contrast, AHT induced an instant anisotropy increase similar to what was found with T-15Ap, suggesting that AHT does not affect binding at Exosite II and probably binds to a third site of α-thrombin other than Exosite I and II.

### Example 5

Quick Evaluation of Binding Constants of Protein-Protein Interactions.

Using the aptamer/thrombin system with known thermodynamic properties, it is possible to obtain the dissociation constant (K_{d}) of the protein-protein binding reactions by taking one single fluorescence measurement in the competitive assay. This capability was demonstrated by calculating K_{d} of α-thrombin/HirF binding reaction to be ˜190 nM using a reported 15mer aptamer-thrombin K_{d/AT} of 75 nM. This Kd is close to reported (150 nM) (Tasset, D. M. et al., (1997) J. Mol. Biol. 272, 688-698.).

### Example 6

Kinetics of Protein-Protein Interactions in Competitive Assays.

While the thermodynamic properties of the protein-protein interactions will probably not be affected by the competitive binding of the aptamer, the reaction rates are most likely still dependent on the kinetics of aptamer-protein binding. The detection of protein-protein interactions where the aptamer is displaced consists of two major steps, the dissociation of the aptamer and thrombin, and the association of thrombin and the prey protein. The affinities of the aptamer and the prey protein for thrombin can be represented by their binding constants:

K_{Apt-Thr}=k₁/k₋₁

K_{Thr-P}=k₂k₋₂

where Apt, Thr and P are designated to aptamer, thrombin and the prey protein, respectively.

One situation that should be considered in the aptamer-based competitive assay is that even though the two binding constants could be very close, there could still be large differences between k₋₁ and k₋₂, and k, and k₂. In the cases where k₋₁<<k₋₂, namely the “off” rate of aptamer is vastly smaller than that of the prey protein, it may take an enormously long

$$Apt - Thr {{}^{{}^{{k}_{- 1}}}}_{{k}_{1}} Apt + Thr$$

$$Thr + P {{}^{{}^{{k}_{2}}}}_{{k}_{- 2}} Thr - P$$

time to detect a signal change even though thermodynamically the protein should be able to displace the aptamer from thrombin.

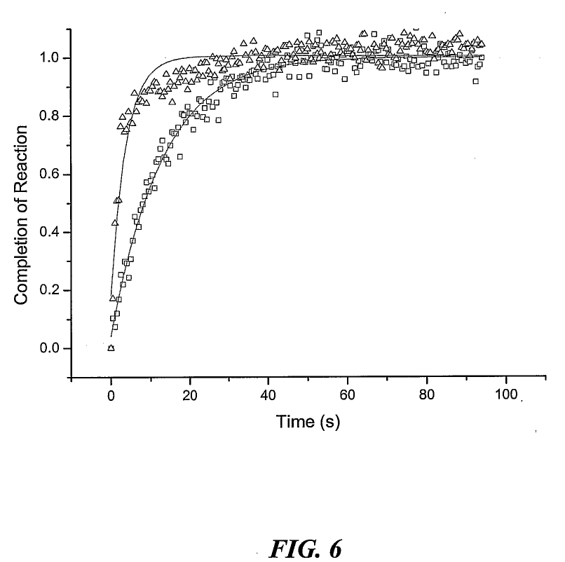

In order to evaluate the possibilities of such false negatives in the assays, a comparison of the reaction rates of aptamer and prey proteins with thrombin were conducted. One direct way to conduct the comparison is to change the order the aptamer and the prey protein are incubated with thrombin. In one experiment, the prey protein was incubated with thrombin first and then the aptamer was used to displace the prey protein from the thrombin/protein complex. In another experiment, the order of adding aptamer and prey protein to thrombin was reversed. By comparing kinetic profiles of these two experiments, it is possible to find out if aptamer binding makes interaction between thrombin and prey protein difficult to take place. HirF was tested along with FQ-15Ap in this way because they compete for the same Exosite I on thrombin. It was found that aptamer replacing HirF was even slower than HirF replacing aptamer (FIG. 6), meaning “off” rate of aptamer would not be so slow as to affect thrombin/HirF interaction.

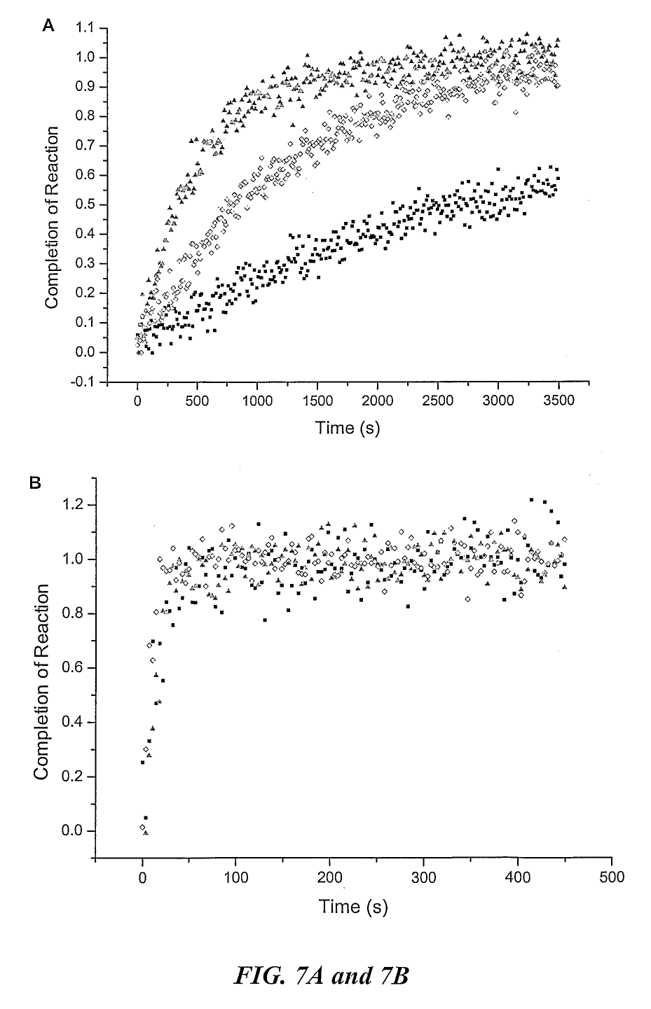

Another indirect method was also used to study the effects of aptamer on thrombin/protein interactions. If aptamer binding to and dissociation from thrombin was a much slower process than thrombin/protein interaction, then changing prey protein concentration would not change the observed rates of thrombin/protein binding in the aptamer-based assay since the prey protein was not in the rate-limiting step of the two steps mentioned earlier. On the other hand, changing aptamer concentration should greatly affect the observed rates since aptamer was in the rate-limiting step. Experiments were conducted to study the thrombin/AT3 interaction. Different concentrations of AT3 were added to thrombin/T-15Ap incubation solution and the anisotropy of the aptamer was monitored as AT3 would displace T-15Ap. The results show a clear dependence of thrombin/AT3 kinetics on AT3 concentration (FIG. 7A), which contradicts the assumption that aptamer binding was the rate limiting step. In another study with AHT, T-15Ap concentration was varied to see if the aptamer had any effects on the observed rates of thrombin/AHT reaction even though T-15Ap and AHT were found to bind to different parts of thrombin (FIG. 7B). The results show no noticeable change in the kinetics, indicating aptamer had no effects on thrombin/AHT binding either.

The protein-protein interactions were not affected by the aptamer binding to its target. Compared to two interacting proteins, aptamers are usually much smaller than their target proteins and tend to bind to the targets only through non-covalent forces. They are also less likely to cause induced conformational changes of the target proteins than in protein-protein interactions.

### Example 7

Protein-Targeted Drugs

Aptamers were first developed as inhibitors of target proteins. The inhibition of proteins of great biological significance may be an important step in dealing with diseases such as cancers. Therefore, aptamers may be clinically useful in the treatment of those diseases. Traditional organic molecule based drugs, when targeted at certain biomolecules, are difficult to control in terms of when and where the intended inhibition should happen.

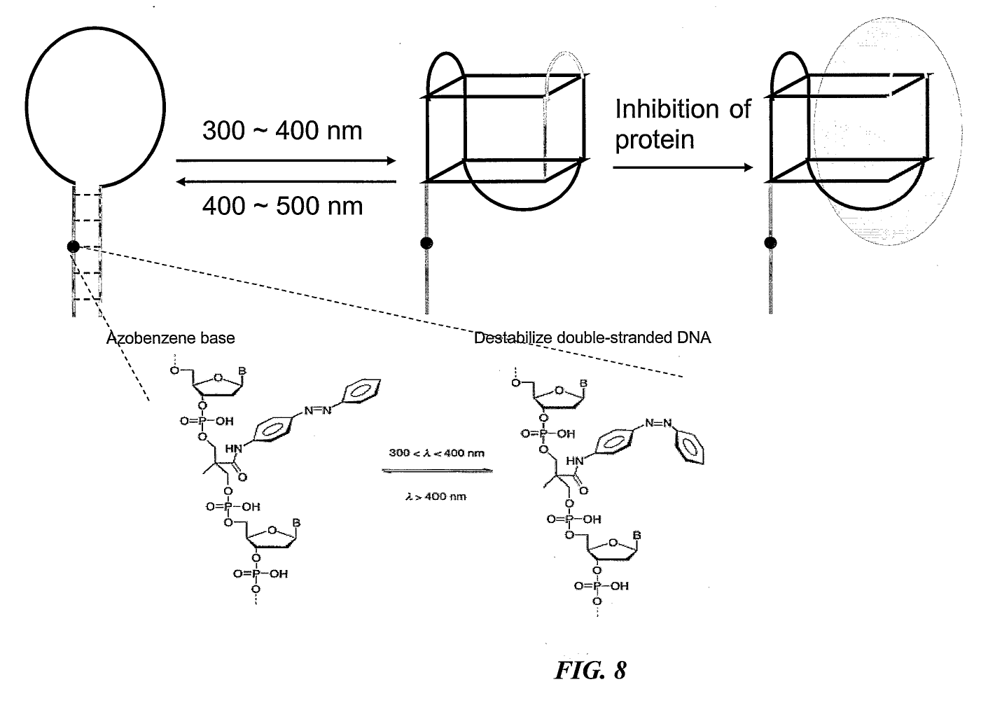

In order to overcome such problems, the following system and compositions were designed to allow controllable release of functional aptamers as protein-targeted drugs. Azobenzene can switch between two of its conformations under light of different wavelengths (light of ˜350 nm for cis-formation and light of >400 nm for tran-formation). See FIG. 8. When incorporated into a double-stranded DNA as an artificial nucleic acid base, the azobenzene could destabilize the double strand in the cis-formation while keep the hybridization intact in tran-formation. This makes it possible to use light to control the hybridization of two DNA strands.

A short strand of DNA was added to one end of an aptamer. This strand is complementary to the other end of the aptamer and contains an azobenzene base, to control when an internal hybridization can take place using light of certain wavelengths. The presence of an internal hybridization prevents the aptamer from forming the conformation that is necessary to bind to its target molecule. By controlling aptamer conformation with light, we can then control whether the target protein can function properly. Another approach of manipulating aptamer conformation would be to use complementary DNA (cDNA) of the whole aptamer sequence that contains one or more azobenzene bases. The intra-molecular DAN hybridization can also be controlled by light in the same way. This novel method provides a convenient way of controlling biological functions using an external physical force.

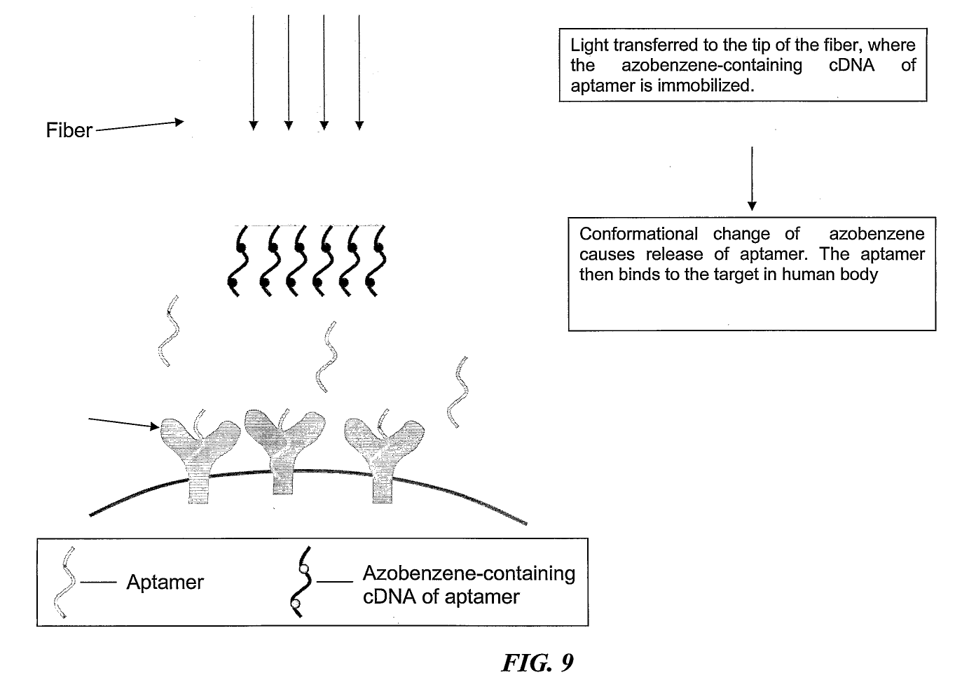

Due to the potential of aptamers as protein-targeted drugs, this method provides convenient ways for controlled drug delivery and release. One approach could be based on an endoscope-like medical device that contains an optical fiber. See for example, FIG. 9. On the tip of the fiber or object that is attached to the fiber, the azobenzene-containing cDNA of aptamers can be immobilized and aptamer molecules can be hybridized with the cDNA. The fiber can then be delivered to the targeted organs in a patient's body. When light of proper wavelength is transferred to the fiber tip inside the body, the conformation of the cDNA can be changed so that hybridization between aptamer and cDNA becomes unstable and aptamer will be released onto the targeted organ. As a result, very precise localized drug delivery can be achieved, which improves the effectiveness of the medicine as well as reduces side-effects caused to other parts of the human body. Another approach for controlled drug delivery will be using a two-photon laser system. In such a system, the highly focused high power laser of longer wavelength causes the molecule to absorb two photons almost simultaneously so that the energy absorbed equals to that from a single photon of shorter wavelength. For example, a 700 nm two-photon laser will act like a 350 nm light source when used as an excitation light. Since light of long wavelengths (infrared or near infrared) can penetrate biological tissues much better than-short wavelengths, a two-photon photon laser system can be used to directly irradiate targeted part in a patient's body from outside. The aptamers hybridized to azobenzene-containing cDNA and previously delivered to inside the patient will be released only in the irradiated region. The benefits gained from controlled drug delivery by using these designs can be crucial in treatment of diseases such as cancers.

### Example 8

Methods for High Selectivity and Affinity Aptamers

Even though aptamers often have high selectivity and affinity for their targets, sometimes greater inhibition of a target is needed. Methods were developed for obtaining even higher selectivity and affinity of the aptamers. During aptamer selection for proteins, usually multiple aptarners would be isolated that may bind to different locations of the protein. Poly(ethylene glycol) (PEG) chain was used to connect two aptamers that bind to different sites of the target protein. The linked double aptamer binds to the protein with much higher affinity because when one of the aptamers comes off of the protein, the other bound aptamer will keep it close to the protein so that it can quickly come back and bind to the protein again. For either one of the two aptamers, the “off” rate of the protein-binding reaction might be the same as in a single aptamer form, but the “on” rate is much higher due to the restriction provided by the other aptamer. Therefore, the equilibrium is greatly shifted to the binding side for both aptamers. The enhanced affinity leads to better inhibition of the protein. As a result, even if only one of the aptamers can inhibit the protein's biological activity, the dual-aptamer still exhibits much higher inhibition. This concept has been demonstrated on human α-thrombin.

Human α-thrombin is a protease that has two aptamers, only one of which inhibits the enzymatic activity of thrombin. The 15mer aptamer called 15Ap has the protein-inhibition capability, while the 27mer 27Ap does not. A short poly(ethylene) glycol (PEG) chain with 18 atoms on the backbone was chosen to be the spacer. Eight units of such spacer were used to link the two aptamers to form DA-8S. Tests of the ability of 15Ap and DA-8S to inhibit thrombin function were carried out using following materials and procedures.

A commonly used test for thrombin activity is based on its ability to cleave a protein called fibrinogen to produce fragments known as fibrin. The crosslinking between fibrin molecules can then develop into a polymer network, often resulting in a white insoluble. This process can be easily monitored with light scattering since insolubles cause more scattered light.

Fibrinogen was from Sigma-Aldrich, Inc. (St. Louis, Mo.), and human α-thrombin was from Haematologic Technologies Inc. (Essex Junction, Vt.). All DNAs were synthesized and purified by GenoMechanix, LLC (Gainesville, Fla.). Other chemicals were from Sigma-Aldrich (St. Louis, Mo.).

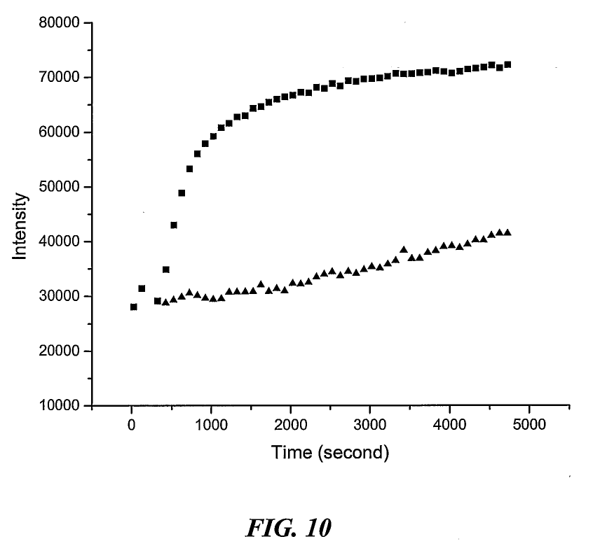

200 μL physiological buffer (20 mM Tris-HCl buffer at pH 7.4, 150 mM NaCl, 5 mM KCl, 1 mM MgCl₂, 1 mM CaCl₂, and 5% V/V glycerol) was added to a 100 μL quartz cuvette placed in a Fluorolog-3 spectrofluorometer (Jobin Yvon Inc., Edison, N.J.). Then 0.5 μL of 100 μM oligonucleotide and 0.5 μL of 10 μM thrombin were added and incubated for 15 minutes. After that, 4 μL of 20 mg/mL fibrinogen was mixed with the solution. The monitoring of scattered light was started sometime before adding fibrinogen. The excitation and emission wavelengths on Fluorolog-3 were both set to 650 mn. Excitation and emission slit widths were 1 nm. The results of scattering light measurements are shown in FIG. 10. It can be seen that DA-8S had a much slower increase of scattering intensity, due to the reduced capability of thrombin to cleave fibrinogen. In our experiments, an enzymatic reaction rate of as much as 10 times slower compared to the 15Ap was observed, indicating that the dual-aptamer concept could really enhance target protein inhibition.

The dual-aptamer approach not only provides better inhibition of targets for potential clinical applications, it may also be found useful in the detection of the targets due to the much improved selectivity and affinity. When labeled with a pair of fluorophores that can have fluorescence resonance energy transfer (FRET) on the two aptamers respectively, the dual-aptamer can bind to its target and display energy transfer because the binding brings the two aptamers, and thus the two fluorophores, close to each other. Because binding of the two aptamers to one protein at the same time is required for the energy transfer to take place, it effectively eliminates false positives caused by non-specific binding in other single aptamer based FRET assays. This approach enables highly selective and sensitive detection of target proteins in complex real-world biological samples.

### Example 9

Diagnosis of Diseases

Human diseases such as cancers often cause overexpression of certain proteins. Sometimes mutated genes would lead to production of proteins that are not found in healthy people. Those proteins can be used as bio-markers for the related diseases. Determination of the levels of the biomarkers will be highly useful for disease diagnoses. Instead of detecting one biomarker for a disease, monitoring multiple biomarkers will provide superior accuracy of diagnosis and offer much more information about the state of the disease.

A method was designed for sensitive and accurate disease diagnosis based on aptamers. This approach will involve development of a panel of aptamers with high affinity for multiple biomarkers of a certain disease. Similar to DNA microchip technology, these aptamers will then be immobilized separately on a solid surface or spotted in a well plate in an array format. Binding of the aptamers with their target molecules will produce detectable signals. This aptamer chip or panel will be standardized by being incubated with extract of tissues or cells from both healthy people and known patients. The signals from all aptamers will be recorded and used to produce distinct patterns for people with and without the disease. Finally, for diagnosis of a patient, the tissue extract from the patient will be incubated with the aptamer panel. The signal pattern generated from the panel will be compared to the standard samples to determine the status of the disease. Because of the high specificity and affinity of aptamers for the biomarkers, the aptamer panel should provide sensitive and accurate diagnoses for many diseases.

### Other Embodimnents

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All references cited herein, are incorporated by reference.

1: An aptamer composition for identifying molecular interactions comprising aptamers which specifically bind a target molecule.

2: The aptamer composition of claim 1, wherein the aptamers are identified by any one of SEQ ID NO's: 1-6.

3: The aptamer composition of claim 2, wherein the aptamers are about 45% homologous to any one of SEQ ID NO's 1-6.

4: The aptamer composition of claim 2, wherein the aptamers are about 55% homologous to any one of SEQ ID NO's 1-6.

5: The aptamer composition of claim 2, wherein the aptamers are about 65 % homologous to any one of SEQ ID NO's 1-6.

6: The aptamer composition of claim 2, wherein the aptamers are about 75% homologous to any one of SEQ ID NO's 1-6.

7: The aptamer composition of claim 2, wherein the aptamers are about 85% homologous to any one of SEQ ID NO's 1-6.

8: The aptamer composition of claim 2, wherein the aptamers are about 95% homologous to any one of SEQ ID NO's 1-6.

9: The aptamer composition of claim 2, wherein the aptamers are about 99% homologous to any one of SEQ ID NO's 1-6.

10: A method for determining molecular interactions comprising:
labeling an aptamer with a fluorophore and/ or quencher;
providing a bait and prey molecule; and,
allowing binding of the aptamer to the bait protein; and,
binding of the bait and prey molecule results in releasing the aptamer resulting in restored fluorescence, thereby determining molecular interactions.

11: The method of claim 10, wherein the aptamers are identified by any one of SEQ ID NO's: 1-6.

12: The method of claim 10, wherein the aptamers are between about 45% to 99% homologous to any one of SEQ ID NO's: 1-6.

13: The method of claim 10, wherein the aptamer is a nucleic acid molecule.

14: The method of claim 10, wherein the aptamer is labeled with a donor molecule at a 5′-end and an acceptor molecule at a 3′-end.

15: The method of claim 14, wherein the donor molecule is a fluorophore molecule.

16: The method of claim 14, wherein the acceptor molecule is a fluorophore quenching molecule.

17: The method of claim 10, wherein binding of the aptamer to bait molecule is detected by a quenching of fluorescence as compared to a baseline fluorescence of unbound aptamer.

18: The method of claim 10, wherein binding of a prey molecule to the bait molecule is detected by displacement of the aptamer as measured by increase in fluorescence as compared to aptamer bound to a bait molecule.

19: The method of claim 10, wherein absence of binding between the bait molecule and the prey molecule is detected by no increase in fluorescence as compared to binding of the aptamer to the bait molecule.

20: A method for determining molecular interactions comprising:
measuring fluorescent anisotropy of an aptamer bound to a target molecule as compared to fluorescent anisotropy of an unbound aptamer;
administering a prey molecule to a composition of aptamer bound to a target molecule; wherein,
binding of the target molecule to the prey molecule dissociates the aptamer-target molecule; and,
measuring changes in anisotropy to determine molecular interaction between target and prey molecules.

21: The method of claim 20, wherein binding of a target molecule to the aptamer increases the fluorescent anisotropy.

22: The method of claim 20, wherein administration of a prey molecule to a composition of aptamer bound to the target molecule dissociates an aptamer-target molecule and decreases fluorescent anisotropy as compared to a complexed aptamer-target molecule anisotropic value.

23: The method of claim 20, wherein anisotropic values are a measure of molecular weight.

24: The method of claim 20, wherein the aptamer molecule is fluorescently labeled.

25: The method of claim 24, wherein the aptamer molecule is fluorescently labeled at a 3′-end.

26: The method of claim 20, wherein comparison of anisotropic values between aptamer alone and aptamer-target molecule complex anisotropic values measured prior to and subsequent to administration of prey molecule.

27: The method of claim 20, wherein affinity between target molecules and prey molecules is a measure of fluorescence.

28: The method of claim 20, wherein identification of candidate protein-protein binding is determined by comparing dissociation constants (K_{D}) between an aptamer and a target/bait molecule.

29: The method of claim 20, wherein the K_{D} of aptamer-target is less than the K_{D} of target-prey.

30: The method of claim 20, wherein the aptamer sequence is altered by specific base changes to alter the KD of an aptamer-target molecule.

31: The method of claim 20, wherein the prey molecule is selected from the group consisting of protein, organic molecule, and nucleic acid molecules.

32: A method of increasing selectivity and affinity of aptamers for a target molecule comprising:
producing aptamers specific for a target molecule;
selecting aptamers that bind different epitopes on the target molecule;
linking the selected aptamers with a linking molecule; thereby, increasing the selectivity and affinity of the aptamers for a target molecule.

33: The method of claim 32, wherein at least two aptamers specific for the target protein are linked.

34: The method of claim 32, wherein the aptamers are linked via a polyethylene glycol chain.

35: The method of claim 32, wherein binding association constants (on rates) of the linked aptamers is greater than the binding association constants of each individual aptamer.

36: The method of claim 32, wherein at least one of the aptamers is labeled with a donor molecule at a 5′-end and an acceptor molecule at a 3′-end.

37: The method of claim 36, wherein the donor molecule is a fluorophore molecule.

38: The method of claim 36, wherein the acceptor molecule is a fluorophore quenching molecule.

39: The method of claim 32, wherein binding of the aptamers to a target molecule is detected by a quenching of fluorescence as compared to a baseline fluorescence of unbound aptamer(s).

40: A method of diagnosing a disease comprising:
binding of an aptamer to a biomarker of disease;
detecting the binding of the aptamer to the biomarker as compared to a control; and,
diagnosing a disease.
